(19) United States
(12) Patent Application Publication
(10) Pub. No.: US 2023/0365618 A1
(43) Pub. Date: Nov. 16, 2023

(54) CRYSTALLINE THIOCHOLESTEROL

(71) Applicant: Durect Corporation, Cupertino, CA (US)

(72) Inventors: ANDREW R. MIKSZTAL, Palo Alto, CA (US); WEIQI LIN, Emerald Hills, CA (US); MICHAEL HOLTZ-MULHOLLAND, Lachine, Quebec (CA); JULIETTE SABBATANI, Montreal, Quebec (CA); TRAVIS LEE HOUSTON, Lafayette, IN (US); STEPHAN D. PARENT, West Lafayette, IN (US)

(21) Appl. No.: 18/028,460

(22) PCT Filed: Oct. 11, 2021
(86) PCT No.: PCT/US2021/054430

§ 371 Date: Mar. 24, 2023

Related U.S. Application Data
(60) Provisional application No. 63/090,619, filed on Oct. 12, 2020.

(51) Int. Cl. C07J 31/00

(52) U.S. Cl. CPC C07J 31/00; C07B 2200/13

(57) ABSTRACT

Crystalline forms of thiocholesterol are disclosed herein. Such forms include crystalline Form thiocholesterol, crystalline Form B thiocholesterol, crystalline thiocholesterol Form C. Processes for making crystalline thiocholesterol, pharmaceutical compositions comprising crystalline thiocholesterol, methods of treatment comprising administering crystalline thiocholesterol, including ctystalline thiocholesterol Form A, crystalline thiocholesterol Form B, and/or crystalline thiocholesterol Form C are further provided.

### CROSS REFERENCE TO RELATED APPLICATIONS

This application claims priority to U.S. Provisional Application No. 63/090,619, filed Oct. 12, 2020, which is incorporated herein by reference.

### BACKGROUND

It has been shown previously that nuclear cholesterol metabolite, (3 S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a] phenanthrene-3-thiol (“Thiocholesterol”), decreases lipid biosynthesis and increases cholesterol secretion and degradation, and may be useful for the treatment and prevention of hypercholesterolemia, hypertriglyceridemia, and conditions related to fat-accumulation and inflammation (e.g. non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, and atherosclerosis).

Cholesterol is used by the body for the manufacture and repair of cell membranes, and the synthesis of steroid hormones and vitamin D, and is transformed to bile acids in the liver. There are both exogenous and endogenous sources of cholesterol. The average American consumes about 450 mg of cholesterol each day and produces an additional 500 mg to 1,000 mg in the liver and other tissues. Another source is the 500 mg to 1,000 mg of biliary cholesterol that is secreted into the intestine daily, and about 50 percent is reabsorbed (enterohepatic circulation).

High serum lipid levels (hypercholesterolemia and hypertriglyceridemia) are associated with the accumulation of cholesterol in arterial walls, and can result in NAFLD and atherosclerosis. The plaques that characterize atherosclerosis inhibit blood flow and promote clot formation, and can ultimately cause death or severe disability via heart attacks and/or stroke. A number of therapeutic agents for the treatment of hyperlipidemia have been developed and are widely prescribed by physicians. Unfortunately, only about 35% of patients are responsive to the currently available therapies.

Non-alcoholic fatty liver disease (NAFLD) is the most common liver disease in the United States. This condition is associated with obesity, type-II adult onset diabetes, sedentary lifestyle, and diets high in fat. The earlier stage of NAFLD, fatty liver, is potentially reversible when proper treatment steps are taken. However, left unchecked, it can progress to inflammation of liver cells (non-alcoholic steatohepatitis, or NASH) which is much more difficult to treat. Without treatment, NASH can result in irreversible scarring of liver tissue (steatonecrosis), with the potential to cause cirrhosis, liver failure, and liver cancer.

Crystalline solids are generally more favorable for processing, storage, and stability than non-crystalline amorphous solids, for example. However, energetics may not favor the ready formation of suitable crystalline solids and polymorphism may make creating stable crystalline solids of a particular active pharmaceutical ingredient impractical. Herein, the inventors disclose crystalline thiocholesterol.

### SUMMARY

In some aspects of the present disclosure, crystalline thiocholesterol is provided.

In other aspects of the present disclosure, stable crystalline thiocholesterol is provided.

In additional aspects of the present disclosure, crystalline thiocholesterol Form A, crystalline thiocholesterol Form B, and crystalline thiocholesterol Form C, are provided.

In yet additional aspects of the present disclosure, mixtures of two or more of crystalline thiocholesterol Form A, crystalline thiocholesterol Form B, and crystalline thiocholesterol Form C, are provided.

In other aspects of the present disclosure, methods of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis; comprising administering to a patient in need thereof an effective amount of a compound or pharmaceutical composition thereof of crystalline thiocholesterol are provided.

In further aspects of the present disclosure, pharmaceutical compositions comprising crystalline thiocholesterol and at least one pharmaceutically acceptable excipient are provided.

In further aspects of the present disclosure, pharmaceutical compositions comprising mixtures of two or more of crystalline thiocholesterol Form A, crystalline thiocholesterol Form B, crystalline thiocholesterol Form C, and at least one pharmaceutically acceptable excipient are provided.

In still further aspects of the disclosure, use of one or more of crystalline thiocholesterol Form A, crystalline thiocholesterol Form B, and crystalline thiocholesterol Form C, and optionally one or more pharmaceutically acceptable excipients, for treating a host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis are provided.

In yet additional aspects of the disclosure, processes for making crystalline thiocholesterol, such as crystalline thiocholesterol Form A, crystalline thiocholesterol Form B, and crystalline thiocholesterol Form C, as are the crystalline thiocholesterols made by such processes are provided.

In additional aspects of the disclosure, stable crystalline thiocholesterol, including crystalline thiocholesterol Form A, crystalline thiocholesterol Form B, and crystalline thiocholesterol Form C, are provided.

In further aspects of the disclosure, crystalline thiocholesterol, or a mixture of crystalline thiocholesterol, or a pharmaceutical composition for use as a medicament is provided.

In additional aspects of the disclosure, crystalline thiocholesterol, or a mixture of crystalline thiocholesterol, or a pharmaceutical composition of crystalline thiocholesterol for use in a method for the treatment or prevention of one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis are provided.

### BRIEF DESCRIPTION OF THE FIGURES

FIG. 1 is a peak picked x-ray powder diffraction for crystalline thiocholesterol Form A.

FIG. 2 is a peak picked x-ray powder diffraction for crystalline thiocholesterol Form B.

FIG. 3 is a peak picked x-ray powder diffraction for crystalline thiocholesterol Form C.

FIG. 4 is an Atomic Displacement Ellipsoid of crystalline thiocholesterol Form A.

FIG. 5 is a DSC thermogram for crystalline thiocholesterol Form A.

FIG. 6 is a DVS plot for crystalline thiocholesterol Form A.

FIG. 7 is an Atomic Displacement Ellipsoid of crystalline thiocholesterol Form B.

FIG. 8 is a DSC thermogram for crystalline thiocholesterol Form B.

### DETAILED DESCRIPTION

Crystalline thiocholesterol is readily analyzed by x-ray powder diffraction. An x-ray powder diffraction pattern is an x-y graph with ° 2θ (diffraction angle) on the x-axis and intensity on the y-axis. The pattern contains peaks which may be used to characterize crystalline thiocholesterol. The peaks are usually represented and referred to by their position on the x-axis Unless otherwise specified, peaks are referred to by their position on the x-axis and not their y-axis intensity.

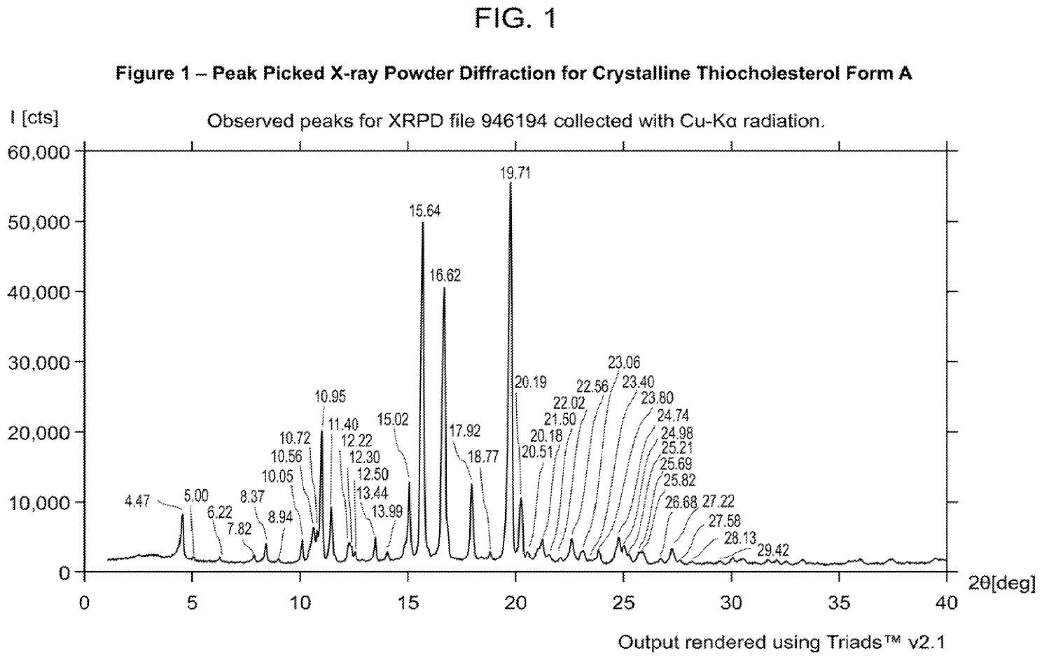

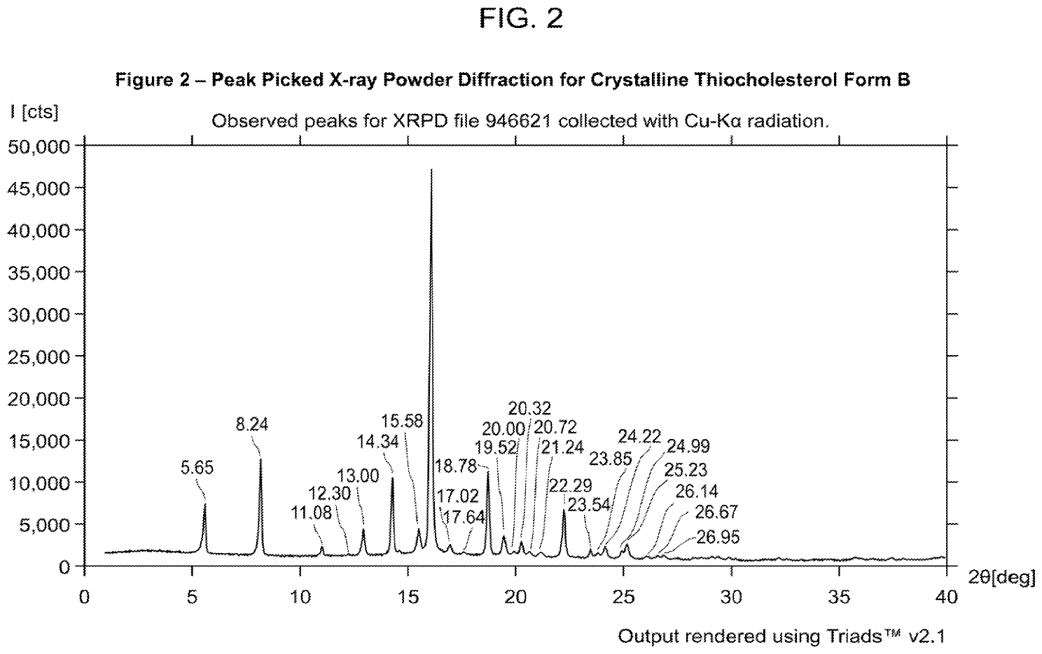

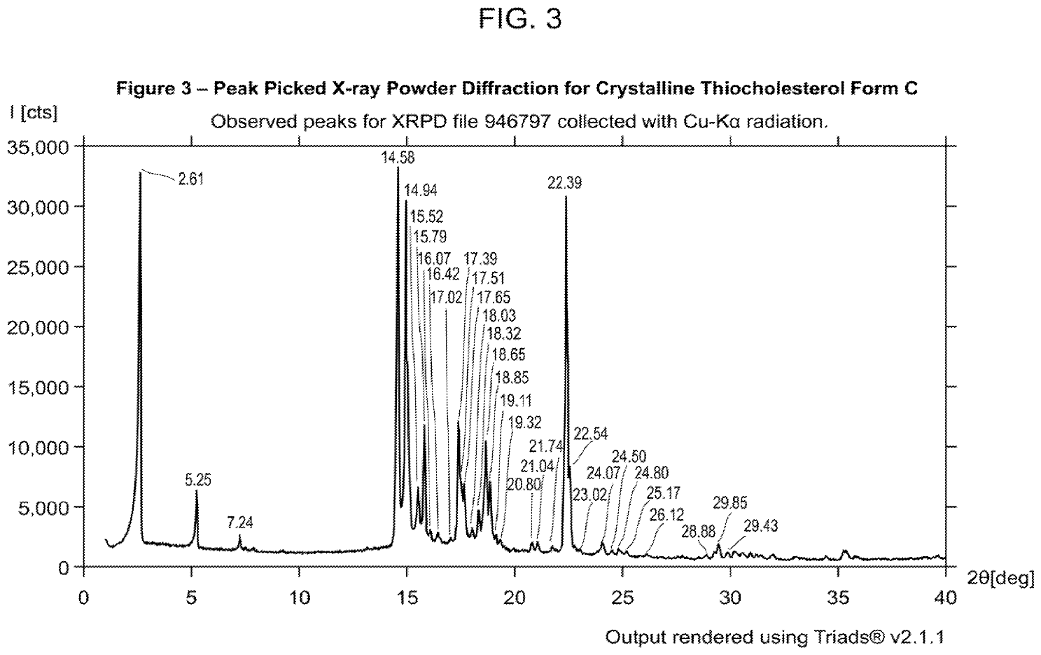

The data from x-ray powder diffraction may be used in multiple ways to characterize crystalline forms. For example, the entire x-ray powder diffraction pattern output from a diffractometer may be used to characterize crystalline thiocholesterol. A smaller subset of such data, however, may also be, and typically is, suitable for characterizing crystalline thiocholesterol. For example, a collection of one or more peaks from such a pattern may be used to characterize crystalline thiocholesterols. As seen in FIG. 3, there is a peak at about 2.6°2θ in crystalline thiocholesterol Form C. The x-ray powder diffraction pattern of crystalline thiocholesterol Form A (FIG. 1) and crystalline thiocholesterol Form B (FIG. 2) do not have a peak at about 2.6°2θ. Thus, the peak at about 2.6°2θ may be used to characterize crystalline thiocholesterol Form C.

In some embodiments, data common to more than one solid form may be used to characterize a collection of such forms. For example, the peak at about 15.6°2θ is common to crystalline thiocholesterol Form A and crystalline thiocholesterol Form B and, because it is within 0.2°2θ of a peak in the x-ray powder diffraction pattern of crystalline thiocholesterol Form C (15.5°2θ), it can also be viewed as common to crystalline thiocholesterol Form C. Thus, a peak between about 15.5°2θ and 15.6°2θ may be used to characterize crystalline thiocholesterol. There are other peaks common to crystalline thiocholesterol, such as a peak between about 5.3°2θ (crystalline thiocholesterol Form C) and about 5.7°2θ (crystalline thiocholesterol Form B). Thus a peak between about 5.3°2θ and about 5.7°2θ may be used to characterize crystalline thiocholesterol other than crystalline thiocholesterol Form A. Likewise, a peak between about 14.9°2θ (crystalline thiocholesterol Form C) and about 15.0°2θ (crystalline thiocholesterol Form A) may be used to characterize crystalline thiocholesterol other than crystalline thiocholesterol Form B.

In the present application, all reported peak values are in ° 2θ with Cu-Kα radiation, e.g., as set forth in Example 7. Indeed, often even a single x-ray powder diffraction peak may be used to characterize such a crystalline form. When crystalline thiocholesterol herein is characterized by “one or more peaks” of an x-ray powder diffraction pattern and such peaks are listed, what is generally meant is that any combination of the peaks listed may be used to characterize crystalline thiocholesterol. Further, the fact that other peaks are present in the x-ray powder diffraction pattern, generally does not negate or otherwise limit that characterization.

In addition to variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, particle size, moisture content, solvent content, instrument parameters, data analysis software, and sample orientation). For example, samples of the same crystalline material prepared under different conditions may yield slightly different diffractograms and different x-ray instruments may operate using different parameters, and these may lead to slightly different diffraction patterns from the same crystalline solid.

Due to such sources of variability, it is common to recite x-ray diffraction peaks using the word “about” prior to the peak value in ° 2θ. For purposes of data reported herein, that value is generally ±0.2°2θ. This generally means that on a well-maintained instrument one would expect the variability in peak measurement to be ±0.2°2θ or less. X-ray powder diffraction peaks cited herein are generally reported with this variability of ±0.2°2θ unless stated otherwise and are generally intended to be reported with such a variability whenever disclosed herein whether the word “about” is present or not, unless context dictates otherwise. Depending on instrument type and calibration, for example, it is possible for an instrument's variability to less than ±0.2°2θ. Accordingly, in certain embodiments, the variability in a peak value or grouping of peak values ±0.1°2θ, or even ±0.05°2θ, rather than ±0.2°2θ.

Thermal methods are another typical technique to characterize solid forms such as salts. Different polymorphs of the same compound often have different endothermic events such as when measured by Differential Scanning calorimetry. Such events may include melting. As with any analytical technique, melting point determinations are also subject to variability. Common sources of variability, in addition to instrumental variability, are due to colligative properties such as the presence of other solid forms or other impurities within a sample whose melting point is being measured. Common variability for thermal measurements is on the order of ±1° C. Thus, unless otherwise specified, the term “about” when used in relation to melting points includes a variability of ±1° C.

Thiocholesterol has the following chemical structure:

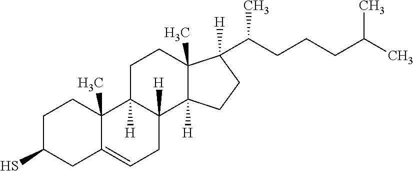

The present disclosure uses the term “Form” to identify different crystalline forms of crystalline thiocholesterol. The differences in the forms can be seen by structure, such as x-ray powder diffraction; properties such as hygroscopicity or thermal behaviors; and/or both. The use of the term “Form A” means crystalline thiocholesterol Form A. Likewise, “Form B” means crystalline thiocholesterol of Form B. Similarly, “Form C”, means crystalline thiocholesterol of Form C.

The generated solids were observed by one or more of polarized light microscopy and x-ray powder diffraction. Materials exhibiting unique crystalline x-ray powder diffraction patterns, based on visual inspection of peaks associated with these materials, were given designations set forth in the present disclosure.

Table 1 summarizes some of the experiments performed on crystalline thiocholesterol Form A to obtain the reported forms of crystalline thiocholesterol in the present disclosure, namely those experiments associated with Example 1 and Example 5.

TABLE 1
A Screen of Thiocholesterol Starting with Crystalline Thiocholesterol
Form A

| | | | | Result |
|---|---|---|---|---|
| Solvent | | Method | Observation | (Form) |
| acetone | (i) | fast evaporation | sheets, lamellae, | B ± 5.3° |
| | | | B single crystals | |
| | (ii) | slurry, ambient, | fines, few thin | A ± 3° |
| | | 14 days | flakes, B | |
| acetonitrile | | slurry, ambient, | fines, B | A ± 3° |
| (ACN) | | 14 days | | |
| dichloromethane | | slow evaporation | off white solids | B |
| (DCM) | | | dendritic/sheet | |
| | | | like soft/waxy | |
| diethyl ether | (i) | slow evaporation | faint yellow | C |
| | | | translucent film, | |
| | | | striations, B | |
| | (ii) | 1. added solvent | 1. clear solution | A |
| | | 2. filtered into | 2. hazy, opaque | disordered |
| | | EtOH | clumps, flakes | |
| | | 3. stored over | forming, B | |
| | | night | 3. fines, flakes, B | |
| ethanol (EtOH) | | slurry, ambient, | fines, B | A ± 3° |
| | | 14 days | | |
| ethyl acetate | (i) | fast evaporation | film, aciculars, B | A ± C |
| (EtOAc) | (ii) | slow cool, 75° C. | spoon shaped, B | A |
| | | stored 12 days in | flat oval solids | Single |
| | | refrigerator | after storage, | crystal |
| | | | single crystals | analysis |
| heptane | | fast evaporation | off white sheets, | A, B, C |
| | | | B areas soft | |
| methanol | (i) | slurry, 40° C., 5 | fines, B | A ± 3° |
| (MeOH) | | days | | |
| | (ii) | filtrate | irregular flakes, | B, C, other |
| | | fast evaporation | blades, and fines | material |
| tetrahydrofuran | | 1. fast | 1. oily waxy | A ± other |
| (THF) | | evaporation | | material |
| | | 2. scratched | 2. nucleated | |
| water | | slurry, ambient, | slow to filter | A ± 3° |
| | | 14 days | fines, B | |
| water/acetone | | slurry, ambient | difficult to filter | A ± 3° |
| 80:20 | | 14 days | fines, B | |
| heptane/toluene | | nitrogen (N₂), | off thin white | B ± C |
| 97:03 | | fast evaporation | lamellae, B | |
| dioxane | | 1. added solvent | 1. clear solution | A, C, other |
| | | 2. filtered into | 2. oiled | material |
| | | water | | |
| | | 3. additional | 3. nucleation | |
| | | dioxane | | |
| | | 4. refrigerated, 2 | 4. — | |
| | | days | | |
| | | 5. freezer, briefly | 5. froze | |
| | | 6. warmed | 6. fines, B flakes, | |
| | | briefly | feathered | |
| | | 7. refrigerated, | 7. solids | |
| | | 12 days | | |
| | | 8. filtered | 8. flakes, B | |

The times, temperatures, and humidity in Table 1 are approximate. In the “Observations” Column, the term “B” means birefringent when a sample is viewed by polarized light microscopy with cross polars. Values provided in the “Result” column are peak positions of additional unknown peaks in the x-ray powder diffraction pattern. Solvent-based methods were used to screen for crystalline thiocholesterol using a diversity of solvents and conditions as set forth in Table 1. Methods using solvents or solvent mixtures included, for example, cooling a solution, evaporation, antisolvent addition, and suspensions (slurries). Variations on these methods can include changes in solvent, solvent mixtures, antisolvent, temperature, cooling rate, concentration, rate of addition, and order of mixing, to name a few possibilities.

Multiple crystalline forms, including stable crystalline forms, of thiocholesterol are herein reported. These are crystalline thiocholesterol Form A, crystalline thiocholesterol Form B, and crystalline thiocholesterol Form C. In the present disclosure, “stable” means that the form does not readily interconvert to another form under a given set of conditions. The term “thermodynamically stable” when comparing forms, means that from a thermodynamic perspective, one form which is more thermodynamically stable than another means it is at a lower energy. This does not necessarily relate to the rate of conversion between forms since the energy of activation between forms may be sufficiently high that a thermodynamically metastable form is sufficiently stable, however, to be of value. For example, diamond is a metastable form of carbon, compared to graphite, but is sufficiently stable to be of commercial value. A metastable form, can, however, so readily convert when exposed to certain conditions. Thus, a form that is stable under one set of conditions (e.g., humidity) may not be stable under another set of conditions. In many embodiments, crystalline thiocholesterol is provided, including stable crystalline thiocholesterol.

In many embodiments, crystalline thiocholesterol Form A is provided. A preparation of crystalline thiocholesterol Form A is set forth in Example 2. Thiocholesterol may be provided as set forth in Example 1. An x-ray powder diffraction pattern for crystalline thiocholesterol Form A is set forth in FIG. 1 which also indicates specifically identified peaks. Table 2 also shows peaks identified in FIG. 1.

TABLE 2
Observed peaks for Crystalline Thiocholesterol Form A

| 2θ (°) | d-spacing (Å) | Intensity (%) |
|---|---|---|
| 4.47 ± 0.20 | 19.770 ± 0.885 | 15 |
| 5.00 ± 0.20 | 17.659 ± 0.706 | 4 |
| 6.22 ± 0.20 | 14.195 ± 0.456 | 4 |
| 7.82 ± 0.20 | 11.298 ± 0.289 | 4 |
| 8.37 ± 0.20 | 10.554 ± 0.252 | 7 |
| 8.94 ± 0.20 | 9.883 ± 0.221 | 3 |
| 10.05 ± 0.20 | 8.799 ± 0.175 | 8 |
| 10.56 ± 0.20 | 8.369 ± 0.158 | 12 |
| 10.72 ± 0.20 | 8.243 ± 0.153 | 11 |
| 10.95 ± 0.20 | 8.076 ± 0.147 | 36 |
| 11.40 ± 0.20 | 7.753 ± 0.136 | 17 |
| 12.22 ± 0.20 | 7.238 ± 0.118 | 7 |
| 12.30 ± 0.20 | 7.192 ± 0.117 | 7 |
| 12.50 ± 0.20 | 7.076 ± 0.113 | 5 |
| 13.44 ± 0.20 | 6.584 ± 0.098 | 9 |
| 13.99 ± 0.20 | 6.327 ± 0.090 | 5 |
| 15.02 ± 0.20 | 5.894 ± 0.078 | 23 |
| 15.64 ± 0.20 | 5.661 ± 0.072 | 90 |
| 16.62 ± 0.20 | 5.329 ± 0.064 | 73 |
| 17.92 ± 0.20 | 4.946 ± 0.055 | 22 |
| 18.77 ± 0.20 | 4.723 ± 0.050 | 5 |
| 19.71 ± 0.20 | 4.500 ± 0.045 | 100 |
| 20.19± 0.20 | 4.394 ± 0.043 | 19 |
| 20.51 ± 0.20 | 4.327 ± 0.042 | 5 |
| 21.18 ± 0.20 | 4.191 ± 0.039 | 8 |
| 21.50 ± 0.20 | 4.130 ± 0.038 | 4 |
| 22.02 ± 0.20 | 4.033 ± 0.036 | 4 |
| 22.56 ± 0.20 | 3.938 ± 0.034 | 8 |
| 23.06 ± 0.20 | 3.853 ± 0.033 | 5 |
| 23.40 ± 0.20 | 3.798 ± 0.032 | 3 |
| 23.80 ± 0.20 | 3.735 ± 0.031 | 5 |
| 24.74 ± 0.20 | 3.596 ± 0.029 | 9 |
| 24.98 ± 0.20 | 3.561 ± 0.028 | 7 |
| 25.21 ± 0.20 | 3.530 ± 0.028 | 5 |
| 25.69 ± 0.20 | 3.464 ± 0.027 | 5 |
| 25.82 ± 0.20 | 3.448 ± 0.026 | 5 |
| 26.68 ± 0.20 | 3.339 ± 0.025 | 3 |
| 27.22 ± 0.20 | 3.273 ± 0.024 | 6 |
| 27.58 ± 0.20 | 3.232 ± 0.023 | 3 |
| 28.13 ± 0.20 | 3.170 ± 0.022 | 2 |
| 29.42 ± 0.20 | 3.033 ± 0.020 | 3 |

Crystalline thiocholesterol Form A is anhydrous and non-hygroscopic with a melting point temperature onset of about 90° C. Crystalline thiocholesterol Form A may be characterized by various analytical techniques, including by x-ray powder diffraction. The x-ray powder diffraction pattern of crystalline thiocholesterol Form A, or portions thereof, may be used to identify crystalline thiocholesterol Form A. Crystalline thiocholesterol Form A contains various x-ray powder diffraction peaks which alone or together may help identify the presence of Form A. For example, in many embodiments, crystalline thiocholesterol Form A may be characterized by an x-ray powder diffraction pattern comprising a peak at about 4.5°2θ. The x-ray powder diffraction pattern may further comprise, for example, one or more peaks at about 8.4°2θ, and at about 11.0°2θ. In these and other embodiments, crystalline thiocholesterol Form A may be characterized by an x-ray powder diffraction pattern comprising a peak at about 4.5°2θ and one or more peaks at about 8.4°2θ, at about 10.1°2θ, at about 11.0°2θ, at about 13.4°2θ, at about 14.0°2θ, at about 15.0°2θ, at about 15.6°2θ, at about 16.6°2θ, at about 17.9°2θ, and at about 19.7°2θ. In many embodiments, crystalline thiocholesterol Form A may be characterized by a peak at about 10.1°2θ. In other embodiments, crystalline thiocholesterol Form A may be characterized by peaks at about 11.0°2θ and at about 15.0°2θ. In these and other embodiments crystalline thiocholesterol Form A may be characterized by (i) an x-ray powder diffraction peak at about 10.1°2θ and one or more peaks at about 4.5°2θ, at about 8.4°2θ, at about 11.0°2θ, at about 12.2°2θ, at about 15.0°2θ, at about 15.6°2θ, at about 16.6°2θ, at about 17.9°2θ, and, at about 19.7°2θ and/or (ii) two x-ray powder diffraction peaks at about 11.0°2θ and at about 15.0°2θ and one or more peaks at about 4.5°2θ, at about 8.4°2θ, at about 10.0°2θ, at about 12.2°2θ, at about 15.6°2θ, at about 16.6°2θ, at about 17.9°2θ, and at about 19.7°2θ. In other embodiments, crystalline thiocholesterol Form A may be characterized by an x-ray powder diffraction pattern substantially the same as that found in FIG. 1.

In these and other embodiments, crystalline thiocholesterol Form A may be characterized by: (i) an x-ray powder diffraction pattern comprising a peak at about 4.5°2θ, a peak at about 11.0°2θ, and a peak at about 15.0°2θ; or (ii) an x-ray powder diffraction pattern comprising a peak at about 4.5°2θ, a peak at about 11.0°2θ, a peak at about 15.0°2θ, a peak at about 15.6°2θ, and a peak at about 16.6°2θ; or (iii) an x-ray powder diffraction pattern comprising a peak at about 4.5°2θ, a peak at about 10.0°2θ, and a peak at about 11.4°2θ; or (iv) an x-ray powder diffraction pattern comprising a peak at about 4.5°2θ, a peak at about 8.4°2θ, and a peak at about 10.0°2θ; or (v) an x-ray powder diffraction pattern comprising a peak at about 4.5°2θ, a peak at about 10.0°2θ, a peak at about 13.4°2θ, and a peak at about 14.0°2θ; or (vi) an x-ray powder diffraction pattern comprising a peak at about 4.5°2θ, a peak at about 10.0°2θ, a peak at about 13.4°2θ, and a peak at about 15.0°2θ; or (vii) an x-ray powder diffraction pattern comprising a peak at about 4.5°2θ, a peak at about 10.0°2θ, a peak at about 13.4°2θ, a peak at about 15.0°2θ, and a peak at about 15.6°2θ; or (viii) an x-ray powder diffraction pattern comprising a peak at about 4.5°2θ, a peak at about 10.0°2θ, a peak at about 15.0°2θ, a peak at about 15.6°2θ, and a peak at about 16.6°2θ; or (ix) an x-ray powder diffraction pattern comprising a peak at about 4.5°2θ, a peak at about 15.0°2θ, a peak at about 15.6°2θ, a peak at about 16.6°2θ, a peak at about 17.9°2θ, and a peak at about 19.7°2θ.

The single-crystal structure of crystalline thiocholesterol Form A is reported herein and may also be used to characterize Form A. The results are presented below in Table 3.

TABLE 3
Crystal Data and Data Collection Parameters for Crystalline Thiocholesterol
Form A

| | |
|---|---|
| Empirical formula | C₂₇H₄₆S |
| Formula weight (g mol⁻¹) | 402.70 |
| Temperature (K) | 299.8(3) |
| Wavelength (Å) | 1.54184 |
| Crystal system | orthorhombic |
| Space group | P2₁2₁2₁ |
| Unit cell parameters | |
| a = 9.1316(3) Å | α = 90° |
| b = 23.8059(6) Å | β = 90° |
| c = 35.2074(8) Å | γ = 90° |
| Unit cell volume (Å³) | 7653.5(4) |
| Cell formula units, Z | 12 |
| Calculated density (g cm⁻³) | 1.048 |
| Absorption coefficient (mm⁻¹) | 1.165 |
| F(000) | 2688 |
| Crystal size (mm³) | 0.2 × 0.11 × 0.04 |
| Reflections used for cell measurement | 6257 |
| θ range for cell measurement | 3.9070°-72.6080° |
| Total reflections collected | 26719 |
| Index ranges | −11 ≤ h ≤ 11; −28 ≤ k ≤ 29; |
| | −26 ≤ l ≤ 44 |
| θ range for data collection | θₘᵢₙ = 3.713°, θₘₐₓ = 78.094° |
| Completeness to θₘₐₓ | 96.8% |
| Completeness to θ_{full} = 67.684° | 99.9% |
| Absorption correction | multi-scan |
| Transmission coefficient range | 0.892-1.000 |
| Refinement method | full matrix least-squares on F² |
| Independent reflections | 14363 [Rᵢₙₜ = 0.0476, R_{σ} = 0.0672] |
| Reflections [ I > 2σ(I) ] | 7471 |
| Reflections/restraints/parameters | 14363/0/775 |
| Goodness-of-fit on F² | S = 0.98 |
| Final residuals [ I > 2σ(I) ] | R = 0.0746, R_{w} = 0.1938 |
| Final residuals [ all reflections ] | R = 0.1230, R_{w} = 0.2381 |
| Largest diff. peak and hole (e Å⁻³) | 0.250, −0.180 |
| Max/mean shift/standard uncertainty | 0.000/0.000 |
| Absolute structure determination | Flack parameter: −0.014(17) |

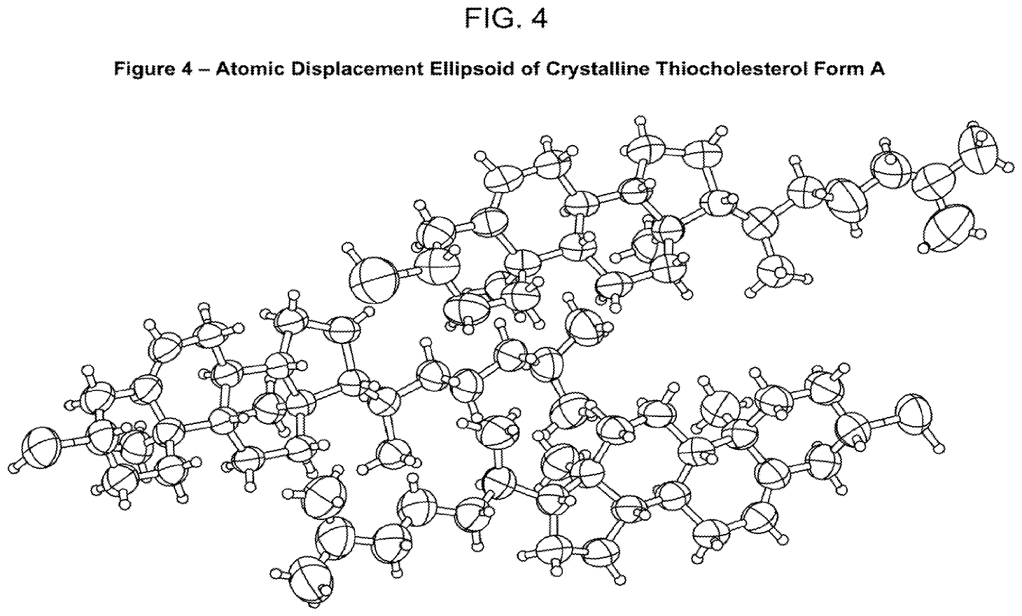

In many embodiments, Form A crystalline thiocholesterol has an orthorhombic crystal system. In these and other embodiments, crystalline thiocholesterol Form A has a space group P2₁2₁. According to the single crystal structure analysis, crystalline thiocholesterol Form A has calculated density of about 1.048 g/cc. In these and other embodiments, crystalline thiocholesterol Form A has following approximate unit cell parameters: a=about 9.13 Å, b=about 23.81 Å, c=about 35.21 Å, α=90°, β=90°, γ=90° with a unit cell volume of about 7654 Å³. In the x-ray powder diffraction patters for crystalline thiocholesterol Form A, there is a broad and weak peak between about 2.0°2θ and about 3.5°2θ which is not attributable to crystalline thiocholesterol Form A. The atomic displacement ellipsoid diagram for crystalline thiocholesterol Form A is found in FIG. 4.

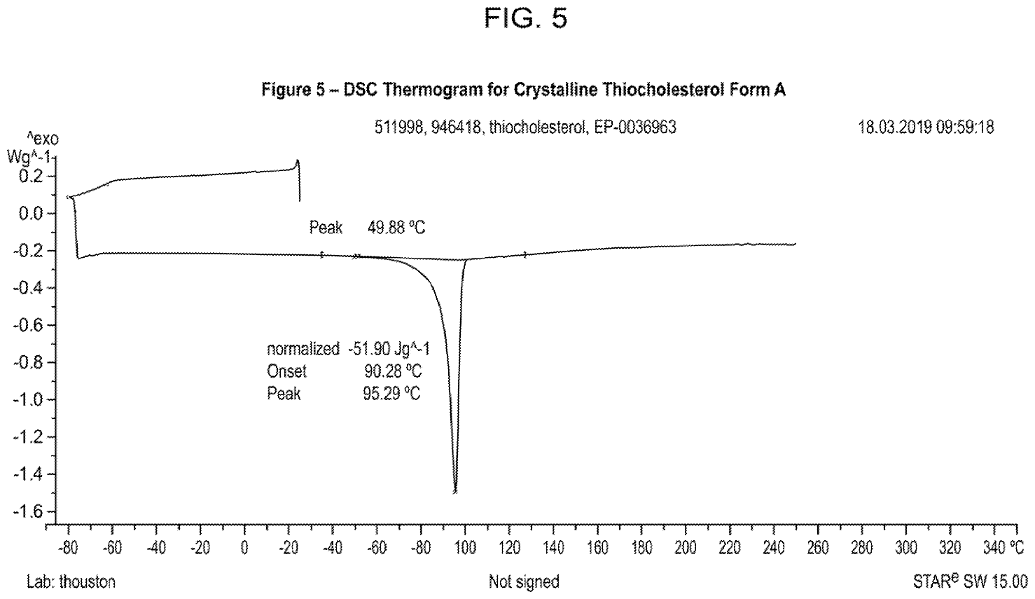

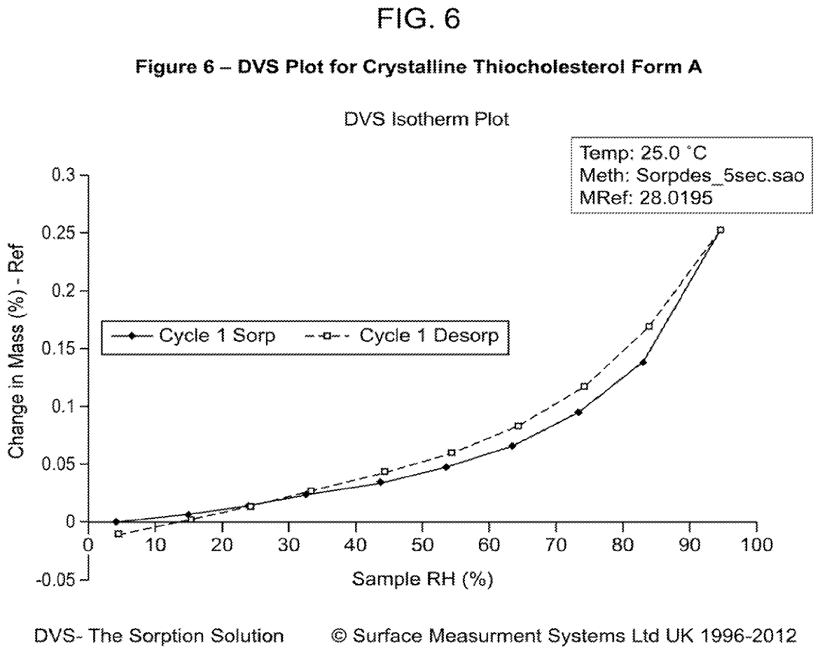

A DSC thermogram for crystalline thiocholesterol Form A is shown in FIG. 5. It indicates a melting point temperature onset of about 90° C. A DVS plot for crystalline thiocholesterol Form A is shown in FIG. 6 indicating that crystalline thiocholesterol Form A has a low hygroscopicity. As seen in FIG. 6, only a small weight change (0.25%) was observed during the DVS cycle and the material recovered from the DVS experiment was identified as crystalline thiocholesterol Form A by x-ray powder diffraction.

Crystalline thiocholesterol Form A may be characterized by a melting point temperature onset of 90° C. in combination with x-ray powder diffraction data. For example, crystalline thiocholesterol Form A may be characterized by a melting point temperature onset of 90° C. and an x-ray powder diffraction pattern comprising a peak at about 4.5°2θ. In other embodiments, crystalline thiocholesterol Form A may be characterized by a melting point temperature onset of about 90° C. and one or more x-ray powder diffraction peaks at about 8.4°2θ, at about 11.0°2θ, at about 13.4°2θ, at about 14.0°2θ, at about 15.0°2θ, at about 15.6°2θ, at about 16.6°2θ, at about 17.9°2θ, and at about 19.7°2θ. In other embodiments, crystalline thiocholesterol Form A may be characterized by a melting point temperature onset of about 90° C. and an x-ray powder diffraction peak at about 10.0°2θ, or by two x-ray powder diffraction peaks at about 11.0°2θ and at about 15.0°2θ. In these and other embodiments crystalline thiocholesterol Form A may be characterized by a melting point temperature onset of about 90° C. and (i) an x-ray powder diffraction peak at about 10.0°2θ and one or more peaks at about 4.5°2θ, at about 8.4°2θ, at about 11.0°2θ, at about 12.2°2θ, at about 15.0°2θ, at about 15.6°2θ, at about 16.6°2θ, at about 17.9°2θ, and at about 19.7°2θ and/or (ii) two x-ray powder diffraction peaks at about 11.0°2θ and at about 15.0°2θ and one or more peaks at about 4.5°2θ, at about 8.4°2θ, at about 10.0°2θ, at about 12.2°2θ, at about 15.6°2θ, at about 16.6°2θ, at about 17.9°2θ, and at about 19.7°2θ.

In these and other embodiments, crystalline thiocholesterol Form A may be characterized by a melting point onset temperature of about 90° C. and (i) an x-ray powder diffraction pattern comprising a peak at about 4.5°2θ, a peak at about 11.0°2θ, and a peak about 15.0°2θ; or (ii) an x-ray powder diffraction pattern comprising a peak at about 4.5°2θ, a peak at about 11.0°2θ, a peak at about 15.0°2θ, a peak at about 15.6°2θ, and a peak at about 16.6°2θ; or (iii) an x-ray powder diffraction pattern comprising a peak at about 4.5°2θ, a peak at about 10.0°2θ, and a peak at about 11.4°2θ; or (iv) an x-ray powder diffraction pattern comprising a peak at about 4.5°2θ, a peak at about 8.4°2θ, and a peak at about 10.0°2θ; or (v) an x-ray powder diffraction pattern comprising a peak at about 4.5°2θ, a peak at about 10.0°2θ, a peak at about 13.4°2θ, and a peak at about 14.0°2θ; or (vi) an x-ray powder diffraction pattern comprising a peak at about 4.5°2θ, a peak at about 10.0°2θ, a peak at about 13.4°2θ, and a peak at about 15.0°2θ; or (vii) an x-ray powder diffraction pattern comprising a peak at about 4.5°2θ, a peak at about 10.0°2θ, a peak at about 13.4°2θ, a peak at about 15.0°2θ, and a peak at about 15.6°2θ; or (viii) an x-ray powder diffraction pattern comprising a peak at about 4.5°2θ, a peak at about 10.0°2θ, a peak at about 15.0°2θ, a peak at about 15.6°2θ, and a peak at about 16.6°2θ; or (ix) an x-ray powder diffraction pattern comprising a peak at about 4.5°2θ, a peak at about 15.0°2θ, a peak at about 15.6°2θ, a peak at about 16.6°2θ, a peak at about 17.9°2θ, and a peak at about 19.7°2θ.

Substantially pure crystalline thiocholesterol Form A is further disclosed. “Substantially pure,” as described herein, generally refers to a form herein that is present without any appreciable amounts, other than potentially trace levels of other forms of crystalline thiocholesterol. Examples of trace levels include not more than about 10%, 5%, 2%, 1.5%, 1%, 0.5%, 0.25%, 0.1%, or less in total relative to the total amount of crystalline thiocholesterol present (measured by weight).

In many embodiments, crystalline thiocholesterol Form B is provided. Example 3 sets forth a preparation of crystalline thiocholesterol Form B. An x-ray powder diffraction pattern for crystalline thiocholesterol Form B is set forth in FIG. 2 and Table 4, which also indicates specifically identified peaks.

TABLE 4
Observed peaks for Crystalline Thiocholesterol Form B

| 2θ (°) | d-spacing (Å) | Intensity (%) |
|---|---|---|
| 5.65 ± 0.20 | 15.632 ± 0.553 | 15 |
| 8.24 ± 0.20 | 10.717 ± 0.260 | 27 |
| 11.08 ± 0.20 | 7.977 ± 0.143 | 5 |
| 12.30 ± 0.20 | 7.193 ± 0.117 | 3 |
| 13.00 ± 0.20 | 6.804 ± 0.104 | 9 |
| 14.34 ± 0.20 | 6.173 ± 0.086 | 22 |
| 15.58 ± 0.20 | 5.685 ± 0.073 | 9 |
| 16.15 ± 0.20 | 5.485 ± 0.067 | 100 |
| 17.02 ± 0.20 | 5.207 ± 0.061 | 5 |
| 17.64 ± 0.20 | 5.022 ± 0.056 | 3 |
| 18.78 ± 0.20 | 4.722 ± 0.050 | 24 |
| 19.52 ± 0.20 | 4.544 ± 0.046 | 7 |
| 20.00 ± 0.20 | 4.437 ± 0.044 | 3 |
| 20.32 ± 0.20 | 4.367 ± 0.043 | 6 |
| 20.72 ± 0.20 | 4.284 ± 0.041 | 3 |
| 21.24 ± 0.20 | 4.179 ± 0.039 | 3 |
| 22.29 ± 0.20 | 3.984 ± 0.035 | 14 |
| 23.54 ± 0.20 | 3.777 ± 0.032 | 4 |
| 23.85 ± 0.20 | 3.727 ± 0.031 | 3 |
| 24.22 ± 0.20 | 3.672 ± 0.030 | 5 |
| 24.99 ± 0.20 | 3.560 ± 0.028 | 3 |
| 25.23 ± 0.20 | 3.528 ± 0.028 | 5 |
| 26.14 ± 0.20 | 3.407 ± 0.026 | 2 |
| 26.67 ± 0.20 | 3.340 ± 0.025 | 2 |
| 26.95 ± 0.20 | 3.306 ± 0.024 | 2 |

Crystalline thiocholesterol Form B is anhydrous with a melting point temperature onset of about 80° C. Crystalline thiocholesterol Form B was prepared in accordance with Table 1 and Example 3. Crystalline thiocholesterol Form B of crystalline thiocholesterol may be characterized by various analytical techniques, including by x-ray powder diffraction. The x-ray powder diffraction pattern of crystalline thiocholesterol Form B, or portions thereof, may be used to identify Form B. Crystalline thiocholesterol Form B contains various x-ray powder diffraction peaks which alone or together may help identify the presence of Form B. For example, in many embodiments, crystalline thiocholesterol Form B may be characterized by an x-ray powder diffraction pattern comprising a peak at about 5.7°2θ. The x-ray powder diffraction pattern may further comprise, for example, one or more peaks at about 8.2°2θ and about 14.3°2θ. In these and other embodiments, crystalline thiocholesterol Form B may be characterized by an x-ray powder diffraction pattern comprising a peak at about 5.7°2θ and one or more peaks at about 8.2°2θ, at about 11.1°2θ, at about 12.3°2θ, at about 13.0°2θ, at about 14.3°2θ, at about 15.6°2θ, at about 16.2°2θ, and at about 18.8°2θ.

In these and other embodiments, crystalline thiocholesterol Form B may be characterized by: (i) an x-ray powder diffraction pattern comprising a peak at about 5.7°2θ, a peak at about 8.2°2θ, a peak at about 14.3°2θ, and a peak at about 16.2°2θ; or (ii) an x-ray powder diffraction pattern comprising a peak at about 5.7°2θ, a peak at about 8.2°2θ, a peak at about 11.1°2θ, a peak at about 13.0°2θ, and a peak at about 14.3°2θ.

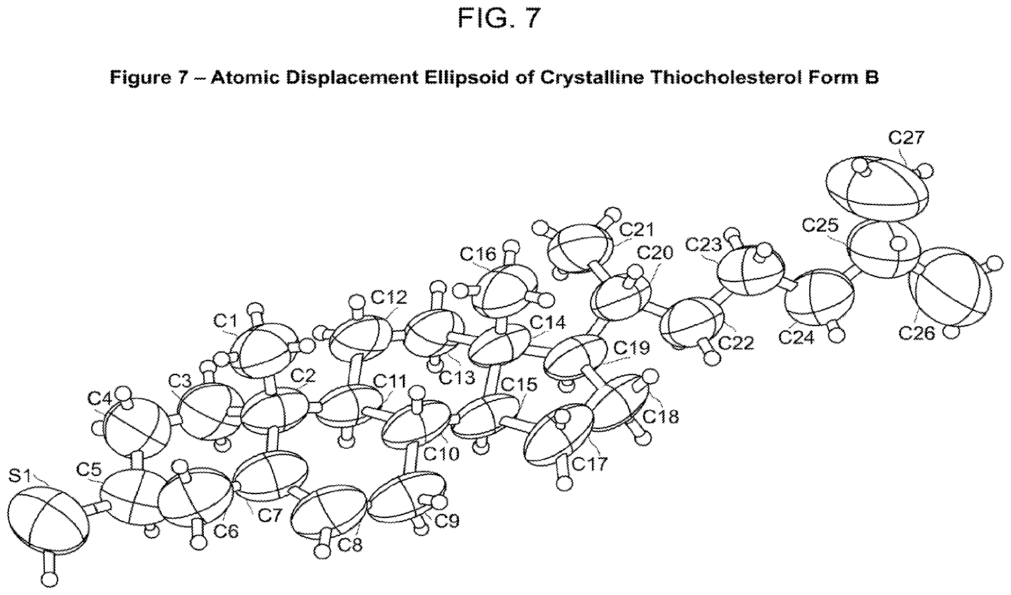

The single-crystal structure of crystalline thiocholesterol Form B is reported herein and may also be used to characterize Form B. The results are presented below in Table 5 with FIG. 7 being an atomic displacement ellipsoid figure.

TABLE 5
Crystal Data and Data Collection Parameters for Crystalline Thiocholesterol
Form B

| Empirical formula | C₂₇H₄₆S |
|---|---|
| Formula weight (g mol⁻¹) | 402.70 |
| Temperature (K) | 298(2) |
| Wavelength (Å) | 1.54184 |
| Crystal system | monoclinic |
| Space group | P2₁ |
| Unit cell parameters | |
| a = 9.1316(3) Å | α = 90° |
| b = 23.8059(6) Å | β = 104.239(6)° |
| c = 35.2074(8) Å | γ = 90° |
| Unit cell volume (Å³) | 1295.97(12) |
| Cell formula units, Z | 2 |
| Calculated density (g cm⁻³) | 1.032 |
| Absorption coefficient (mm⁻¹) | 1.146 |
| F(000) | 448 |
| Crystal size (mm³) | 0.55 × 0.43 × 0.06 |
| Reflections used for cell measurement | 2717 |
| θ range for cell measurement | 4.4160°-75.0730° |
| Total reflections collected | 5888 |
| Index ranges | −13 ≤ h ≤ 13; −9 ≤ k ≤ 5; |
| | −19 ≤ l ≤ 20 |
| θ range for data collection | θₘᵢₙ = 4.133°, θₘₐₓ = 77.308° |
| Completeness to θₘₐₓ | 95.7% |
| Completeness to θ_{full} = 67.684° | 99.5% |
| Absorption correction | multi-scan |
| Transmission coefficient range | 0.678-1.000 |
| Refinement method | full matrix least-squares on F² |
| Independent reflections | 3694 [Rᵢₙₜ = 0.0240, R_{σ} = 0.0355] |
| Reflections [ I > 2σ(I) ] | 2478 |
| Reflections/restraints/parameters | 3694/1/260 |
| Goodness-of-fit on F² | S = 1.08 |
| Final residuals [ I > 2σ(I) ] | R = 0.0591, R_{w} = 0.1880 |
| Final residuals [ all reflections ] | R = 0.0740, R_{w} = 0.2067 |
| Largest diff. peak and hole (e Å⁻³) | 0.172, −0.128 |
| Max/mean shift/standard uncertainty | 0.000/0.000 |

In many embodiments, crystalline thiocholesterol Form B has a monoclinic crystal system. In these and other embodiments, crystalline thiocholesterol Form B has a space group P2₁. According to the single crystal structure analysis, crystalline thiocholesterol Form B has calculated density of about 1.032 g/cc. In these and other embodiments, crystalline thiocholesterol Form B has following approximate unit cell parameters a=about 11.04 Å, b=about 7.56 Å, c=about 16.03 Å, α=90°,β=104.24°, γ=90° with a unit cell volume of about 1296 Å³ In other embodiments, crystalline thiocholesterol Form B may be characterized by an x-ray powder diffraction pattern substantially the same as that found in FIG. 2.

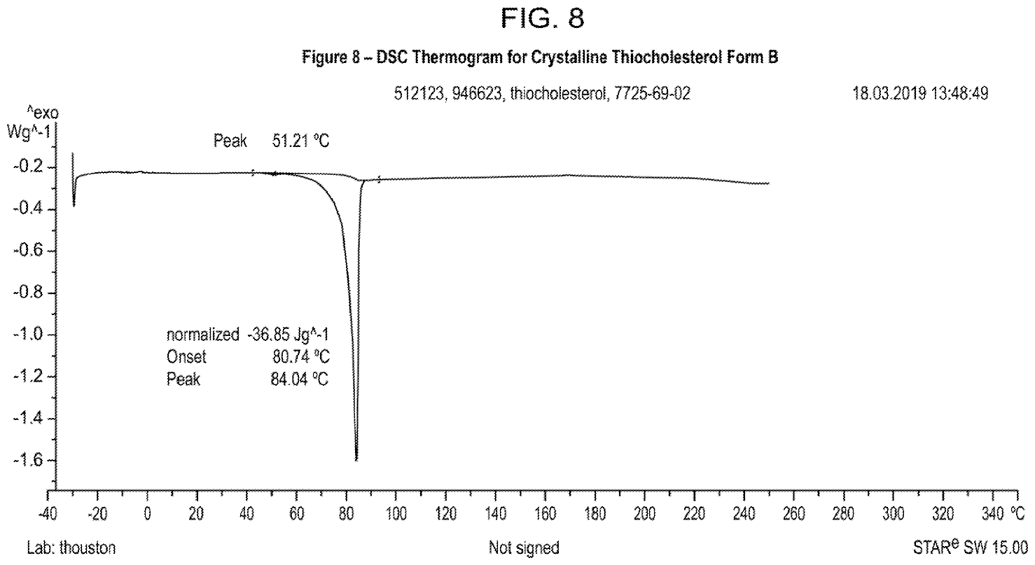

A DSC thermogram for crystalline thiocholesterol Form B is shown in FIG. 8. It indicates a melting point temperature onset of about 80° C. Crystalline thiocholesterol Form B may be characterized by a melting point temperature onset of about 80° C. It may also be characterized by a melting point temperature onset of about 80° C. in combination with x-ray powder diffraction data. For example, crystalline thiocholesterol Form B may be characterized by a melting point temperature onset of about 80° C. and an x-ray powder diffraction pattern comprising a peak at about 5.7°2θ. In other embodiments, crystalline thiocholesterol Form B may be characterized by a melting point temperature onset of about 80° C. and an x-ray powder diffraction pattern comprising a peak at about 5.7°2θ, one or more peaks at about 8.2°2θ, at about 11.1°2θ, at about 13.0°2θ, at about 14.3°2θ, at about 15.6°2θ, at about 16.2°2θ, and at about 18.8°2θ.

In these and other embodiments, crystalline thiocholesterol Form B may be characterized by a melting point onset temperature of about 80° C. and (i) an x-ray powder diffraction pattern comprising a peak at about 5.7°2θ, a peak at about 8.2°2θ, a peak at about 14.3°2θ, and a peak at about 16.2°2θ; or (ii) an x-ray powder diffraction pattern comprising a peak at about 5.7°2θ, a peak at about 8.2°2θ, a peak at about 11.1°2θ, a peak at about 13.0°2θ, and a peak at about 14.3°2θ.

Substantially pure crystalline thiocholesterol Form B is further disclosed. “Substantially pure,” as described herein, generally refers to a form herein that is present without any appreciable amounts, other than potentially trace levels of other forms of crystalline thiocholesterol. Examples of trace levels include not more than about 10%, 5%, 2%, 1.5%, 1%, 0.5%, 0.25%, 0.1%, or less in total relative to the total amount of crystalline thiocholesterol present (measured by weight).

Relative to Crystalline Thiocholesterol Form B, Crystalline Thiocholesterol Form A exhibits a higher crystal density and melt onset (with higher heat of fusion); this suggests that Crystalline Thiocholesterol Form A is more thermodynamically stable than Crystalline Thiocholesterol Form B.

In many embodiments, crystalline thiocholesterol Form C is provided. Example 4 sets forth a preparation for crystalline thiocholesterol Form C. An x-ray powder diffraction pattern for crystalline thiocholesterol Form C is set forth in FIG. 3 and Table 6, which also indicates specifically identified peaks.

TABLE 6
Observed peaks for Crystalline Thiocholesterol Form C

| °2θ | d space (Å) | Intensity (%) |
|---|---|---|
| 2.61 ± 0.20 | 33.770 ± 2.583 | 98 |
| 5.25 ± 0.20 | 16.830 ± 0.641 | 19 |
| 7.24 ± 0.20 | 12.203 ± 0.337 | 8 |
| 14.58 ± 0.20 | 6.070 ± 0.083 | 100 |
| 14.94 ± 0.20 | 5.924 ± 0.079 | 92 |
| 15.52 ± 0.20 | 5.706 ± 0.073 | 20 |
| 15.79 ± 0.20 | 5.608 ± 0.071 | 36 |
| 16.07 ± 0.20 | 5.512 ± 0.068 | 9 |
| 16.42 ± 0.20 | 5.394 ± 0.065 | 9 |
| 17.02 ± 0.20 | 5.206 ± 0.061 | 7 |
| 17.39 ± 0.20 | 5.095 ± 0.058 | 36 |
| 17.51 ± 0.20 | 5.061 ± 0.057 | 22 |
| 17.65 ± 0.20 | 5.021 ± 0.056 | 21 |
| 18.03 ± 0.20 | 4.917 ± 0.054 | 10 |
| 18.32 ± 0.20 | 4.840 ± 0.052 | 14 |
| 18.65 ± 0.20 | 4.754 ± 0.051 | 32 |
| 18.85 ± 0.20 | 4.705 ± 0.049 | 22 |
| 19.11 ± 0.20 | 4.639 ± 0.048 | 8 |
| 19.32 ± 0.20 | 4.591 ± 0.047 | 7 |
| 20.80 ± 0.20 | 4.267 ± 0.041 | 6 |
| 21.04 ± 0.20 | 4.218 ± 0.040 | 6 |
| 21.74 ± 0.20 | 4.085 ± 0.037 | 5 |
| 22.39 ± 0.20 | 3.967 ± 0.035 | 93 |
| 22.54 ± 0.20 | 3.941 ± 0.035 | 25 |
| 23.02 ± 0.20 | 3.861 ± 0.033 | 4 |
| 24.07 ± 0.20 | 3.695 ± 0.030 | 6 |
| 24.50 ± 0.20 | 3.630 ± 0.029 | 4 |
| 24.80 ± 0.20 | 3.587 ± 0.028 | 5 |
| 25.17 ± 0.20 | 3.536 ± 0.028 | 4 |
| 26.12 ± 0.20 | 3.409 ± 0.026 | 3 |
| 28.88 ± 0.20 | 3.089 ± 0.021 | 3 |
| 29.43 ± 0.20 | 3.032 ± 0.020 | 6 |
| 29.85 ± 0.20 | 2.991 ± 0.020 | 4 |

Form C is a crystalline form of thiocholesterol and is likely anhydrous. It was prepared in accordance with Table 1 and also Example 4. Thiocholesterol Form C may be characterized by various analytical techniques, including by x-ray powder diffraction. The x-ray powder diffraction pattern of crystalline thiocholesterol Form C, or portions thereof, may be used to identify Form C. Crystalline thiocholesterol Form C contains various x-ray powder diffraction peaks which alone or together may help identify the presence of Form C. For example, in many embodiments, crystalline thiocholesterol Form C may be characterized by an x-ray powder diffraction pattern comprising a peak at about 2.6°2θ. The x-ray powder diffraction pattern may further comprise, for example, one or more peaks at about 5.3°2θ, about 7.2°2θ, about 14.6°2θ, and about 14.9°2θ. In other embodiments, crystalline thiocholesterol Form C may be characterized by a peak at about 7.2°2θ. In these and other embodiments, crystalline thiocholesterol Form C may be characterized by an x-ray powder diffraction pattern comprising a peak at about 7.2°2θ and one or more peaks at about 2.6°2θ, about 5.3°2θ, about 14.6°2θ, and about 14.9°2θ. In other embodiments, crystalline thiocholesterol Form C may be characterized by an x-ray powder diffraction pattern substantially the same as that found in FIG. 3.

Substantially pure crystalline thiocholesterol Form C is further disclosed. “Substantially pure,” as described herein, generally refers to a form herein that is present without any appreciable amounts, other than potentially trace levels of other forms of crystalline thiocholesterol. Examples of trace levels include not more than about 10%, 5%, 2%, 1.5%, 1%, 0.5%, 0.25%, 0.1%, or less in total relative to the total amount of crystalline thiocholesterol present (measured by weight).

Aspects of the present disclosure also include methods for preparing thiocholesterol. In some embodiments, methods include halogenating cholesterol to produce 3β-halo-5-cholestene; contacting the 3β-halo-5-cholestene with a thiourea to produce a cholestene isothiouronium salt; and hydrolyzing the isothiouronium salt to generate thiocholesterol (Scheme IA):

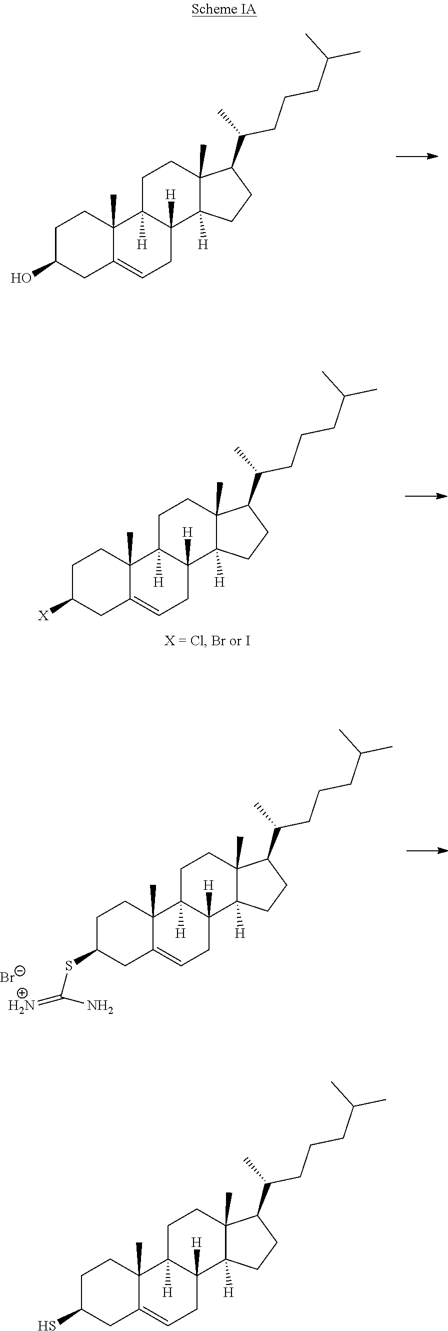

In some embodiments, halogenating cholesterol includes contacting cholesterol with an organophosphorus compound and halogen source. In some embodiments, the organophosphorus compound is triphenylphosphine. In other embodiments, the organophosphorus compound is 1,2-bis(diphenylphosphino)ethane (DPPE). In some instances, a catalytic amount of organophosphorus compound is contacted with cholesterol. In other instances, a stoichiometric amount of organophosphorus compound is contacted with cholesterol, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more and including 10 equivalents or more. In some embodiments, the halogen source is an alkyl halide, such as carbon tetrachloride, carbon tetrabromide or carbon tetraiodide. In certain instances, the halogen source is carbon tetrabromide. In other embodiments, the halogen source is a metal halide, such as lithium bromide. In some instances, a stoichiometric amount of the halogen source is contacted with cholesterol, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more and including 10 equivalents or more. In certain embodiments, methods include contacting cholesterol with 1.0 equivalents of the organophosphorus compound and 1.0 equivalents of the halogen source. In certain instances, methods include contacting cholesterol with triphenylphosphine in carbon tetrabromide.

Halogenating cholesterol may be performed at a temperature which varies, such as from −10° C. to 40° C., such as from −5° C. to 35° C., such as from 0° C. to 30° C. such as from 10° C. to 25° C. and including at about 20° C. In certain embodiments, the halogen source and the organophosphorus compound are contacted with cholesterol at a first temperature and mixed for a predetermined period of time at a second temperature to produce 3β-halogenated cholestene. In some instances, the first temperature is a temperature which ranges from −15° C. to 15° C., such as from −10° C. to 10° C., such as from −5° C. to 5° C. and including at 0 ° C. In some instance, the second temperature is a temperature which ranges from 10° C. to 30° C., such as from 15° C. to 25° C. and including at about 20° C. The organophosphorus compound and halogen source may be mixed with cholesterol for 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 9 hours or more, such as 10 hours or more, such as 11 hours or more and including for 12 hours or more

In practicing the subject methods, the 3β-halogenated cholestene is contacted with a thiourea to produce a cholestene isothiouronium salt. In some embodiments, the thiourea is an unsubstituted thiourea. In other embodiments, the thiourea is a substituted thiourea. In embodiments, the thiourea may be contacted with the 3β-halogenated cholestene at a temperature which varies, such as from −15° C. to 15° C., such as from −10° C. to 10° C., such as from −5° C. to 5° C. and including at 0° C. The thiourea may be mixed with 3β-halogenated cholestene for 0.5 hours or more, such as 1 hour or more, such as 2 hours or more, such as 3 hours or more, such as 4 hours or more, such as 5 hours or more, such as 6 hours or more, such as 7 hours or more, such as 8 hours or more, such as 9 hours or more, such as 10 hours or more, such as 11 hours or more and including for 12 hours or more. The amount of the thiourea contacted with the 3β-halogenated cholestene may vary and may be 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more and including 10 equivalents or more.

To produce thiocholesterol, the cholestene isothiouronium salt is hydrolyzed. In some embodiments, the cholestene isothiouronium salt is hydrolyzed in the presence of a base followed by an aqueous acid workup. In some embodiments, the base is metal hydroxide base, such as potassium hydroxide, sodium hydroxide, lithium hydroxide, sodium methoxide or potassium methoxide. The cholestene isothiouronium salt may be contacted with the base at a temperature that ranges from −10° C. to 30° C., such as from 10° C. to 25° C. and including at about 20° C. In certain embodiments, the base is contacted with cholestene isothiouronium salt at a first temperature and mixed for a predetermined period of time at a second temperature. In some instances, the first temperature is a temperature which ranges from −15° C. to 15° C., such as from −10° C. to 10° C., such as from −5° C. to 5° C. and including at 0° C. In some instance, the second temperature is a temperature which ranges from 10° C. to 30° C., such as from 15° C. to 25° C. and including at about 22° C. The amount of the base contacted with the cholestene isothiouronium salt may vary and may be 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more and including 10 equivalents or more.

Methods according to certain embodiments further include an aqueous acid workup of the hydrolysis product to produce thiocholesterol. In some embodiments, the acid is chosen from acetic acid, hydrochloric acid, citric acid, para-toluene sulfonic acid, formic acid, and methane sulfonic acid.

The components used in each step of the subject methods described herein may be a purified composition or a crude composition as desired. The term “purified” is used in its conventional sense to refer to a composition where at least some isolation or purification process has been conducted, such as for example, filtration or aqueous workup of a reaction mixture. In certain instances, purification includes liquid chromatography, recrystallization, distillation (e.g., azeotropic distillation) or other type of compound purification. In some embodiments, a reaction mixture is used in a subsequent step in the methods described herein as a crude mixture where no purification or other workup of the reaction mixture has been conducted. In certain instances, the crude composition reaction mixtures include the compound of interest in sufficient purity such as where the crude composition includes a compound of interest in a purity of 90% or greater, such as 95% or greater, such as 97% or greater and including 99% or greater, as determined by high performance liquid chromatography (HPLC), proton nuclear magnetic resonance spectroscopy (¹H NMR) or a combination thereof.

This disclosure also relates to pharmaceutical compositions containing crystalline thiocholesterol as disclosed herein. Such pharmaceutical compositions are comprised of one or more pharmaceutically acceptable excipients and crystalline thiocholesterol as set forth in the present disclosure. Such pharmaceutical compositions may be administered orally or configured to be delivered as any effective conventional dosage unit forms, including, for example, immediate, slow and timed-release oral preparations, parenterally, topically, nasally, ophthalmically, optically, sublingually, rectally, vaginally, and the like.

The present disclosure further includes mixtures of forms of crystalline thiocholesterol. For examples, mixtures of two or more of crystalline thiocholesterol Form A crystalline thiocholesterol Form B, and/or crystalline thiocholesterol Form C, are provided. The amount of each form present in such mixtures ranges from, for example, about 0.1% to about 99.9% by weight. Other ranges include about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, and about 0.1% to about 10% by weight. Other ranges include about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, and about 0.1% to about 1% by weight. Additional ranges include about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, and about 0.1% to about 0.2% by weight. Such mixtures may also be present in pharmaceutical compositions for the comprising one or more pharmaceutically acceptable excipients.

The present disclosure further includes methods and uses for treating and/or preventing diseases (e.g., in humans) such as one or more of hypercholesterolemia, hypertriglyceridemia, and conditions related to fat-accumulation and inflammation (e.g., non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, and atherosclerosis) with effective amounts crystalline thiocholesterol and/or pharmaceutical compositions comprising crystalline thiocholesterol of the present disclosure.

The present disclosure may be further described by one or more of the non-limiting clauses that follow.

Clause 1. Crystalline thiocholesterol.

Clause 2. Crystalline thiocholesterol Form A.

Clause 3. Crystalline thiocholesterol Form A of clause 2, having an x-ray powder diffraction pattern comprising a peak at about 4.5°2θ.

Clause 4. Crystalline thiocholesterol Form A of clause 3, having an x-ray powder diffraction pattern further comprising a peak at about 8.4°2θ.

Clause 5. Crystalline thiocholesterol Form A of clause 3, having an x-ray powder diffraction pattern further comprising a peak at about 11.0°2θ.

Clause 6. Crystalline thiocholesterol Form A of clause 2, having an x-ray powder diffraction pattern comprising a peak at about 4.5°2θ and one or more peaks at about 8.4°2θ, at about 10.1°2θ, at about 11.0°2θ, at about 13.4°2θ, at about 14.0°2θ, at about 15.0°2θ, at about 15.6°2θ, at about 16.6°2θ, at about 17.9°2θ, and at about 19.7°2θ.

Clause 7. Crystalline thiocholesterol Form A of clause 6, having an x-ray powder diffraction pattern comprising a peak at about 10.1°2θ.

Clause 8. Crystalline thiocholesterol Form A of clause 6, having an x-ray powder diffraction pattern comprising a peak at about 13.4°2θ

Clause 9. Crystalline thiocholesterol Form A of clause 6, having an x-ray powder diffraction pattern comprising a peak at about 14.0°2θ.

Clause 10. Crystalline thiocholesterol Form A of clause 6, having an x-ray powder diffraction pattern comprising a peak at about 15.0°2θ.

Clause 11. Crystalline thiocholesterol Form A of clause 6, having an x-ray powder diffraction pattern comprising a peak at about 15.6°2θ.

Clause 12. Crystalline thiocholesterol Form A of clause 6, having an x-ray powder diffraction pattern comprising a peak at about 16.6°2θ.

Clause 13. Crystalline thiocholesterol Form A of clause 6, having an x-ray powder diffraction pattern comprising a peak at about 17.9°2θ.

Clause 14. Crystalline thiocholesterol Form A of clause 6, having an x-ray powder diffraction pattern comprising a peak at about 19.7°2θ.

Clause 15. Crystalline thiocholesterol Form A of clause 6, having an x-ray powder diffraction pattern comprising a peak at about 11.0°2θ and a peak at about 15.0°2θ.

Clause 16. Crystalline thiocholesterol Form A of clause 6, having an x-ray powder diffraction pattern comprising a peak at about 11.0°2θ, a peak at about 15.0°2θ, a peak at about 15.6°2θ, and a peak at about 16.6°2θ.

Clause 17. Crystalline thiocholesterol Form A of clause 6, having an x-ray powder diffraction pattern comprising a peak at about 10.1°2θ, and a peak at about 11.4°2θ.

Clause 18. Crystalline thiocholesterol Form A of clause 6, having an x-ray powder diffraction pattern comprising a peak at about 8.4°2θ, and a peak at about 10.0°2θ.

Clause 19. Crystalline thiocholesterol Form A of clause 6, having an x-ray powder diffraction pattern comprising a peak at about 10.1°2θ, a peak at about 13.4°2θ, and a peak at about 14.0°2θ.

Clause 20. Crystalline thiocholesterol Form A of clause 6, having an x-ray powder diffraction pattern comprising a peak at about 10.1°2θ, a peak at about 13.4°2θ, and a peak at about 15.0°2θ.

Clause 21. Crystalline thiocholesterol Form A of clause 6, having an x-ray powder diffraction pattern comprising a peak at about 10.1°2θ, a peak at about 13.4°2θ, a peak at about 15.0°2θ, and a peak at about 15.6°2θ.

Clause 22. Crystalline thiocholesterol Form A of clause 6, having an x-ray powder diffraction pattern comprising a peak at about 10.1°2θ, a peak at about 15.0°2θ, a peak at about 15.6°2θ, and a peak at about 16.6°2θ.

Clause 23. Crystalline thiocholesterol Form A of clause 6, having an x-ray powder diffraction pattern comprising a peak at about 15.0°2θ, a peak at about 15.6°2θ, a peak at about 16.6°2θ, a peak at about 17.9°2θ, and a peak at about 19.7°2θ.

Clause 24. Crystalline thiocholesterol Form A of clause 2, having an x-ray powder diffraction pattern comprising a peak at about 10.1°2θ.

Clause 25. Crystalline thiocholesterol Form A of clause 24, having an x-ray powder diffraction pattern comprising a peak at about 10.1°2θ and one or more peaks at about 4.5°2θ, at about 8.4°2θ, at about 11.0°2θ, at about 12.2°2θ, at about 15.0°2θ, at about 15.6°2θ, at about 16.6°2θ, at about 17.9°2θ, and at about 19.7°2θ.

Clause 26. Crystalline thiocholesterol Form A of clause 24, having an x-ray powder diffraction pattern comprising a peak at about 8.4°2θ.

Clause 27. Crystalline thiocholesterol Form A of clause 24, having an x-ray powder diffraction pattern comprising a peak at about 11.0°2θ.

Clause 28. Crystalline thiocholesterol Form A of clause 24, having an x-ray powder diffraction pattern comprising a peak at about 12.2°2θ.

Clause 29. Crystalline thiocholesterol Form A of clause 24, having an x-ray powder diffraction pattern comprising a peak at about 15.0°2θ.

Clause 30. Crystalline thiocholesterol Form A of clause 24, having an x-ray powder diffraction pattern comprising a peak at about 15.6°2θ.

Clause 31. Crystalline thiocholesterol Form A of clause 24, having an x-ray powder diffraction pattern comprising a peak at about 16.6°2θ.

Clause 32. Crystalline thiocholesterol Form A of clause 24, having an x-ray powder diffraction pattern comprising a peak at about 17.9°2θ.

Clause 33. Crystalline thiocholesterol Form A of clause 24, having an x-ray powder diffraction pattern comprising a peak at about 19.7°2θ.

Clause 34. Crystalline thiocholesterol Form A of any one of clauses 1-2 and 24-33 having an x-ray powder diffraction pattern comprising two peaks at about 11.0°2θ and at about 15.0°2θ and one or more peaks at about 4.5°2θ, at about 8.4°2θ, at about 10.1°2θ, at about 12.2°2θ, at about 15.6°2θ, at about 16.6°2θ, at about 17.9°2θ, and at about 19.7°2θ.

Clause 35. Crystalline thiocholesterol Form A of clause 34, having an x-ray powder diffraction pattern comprising a peak at about 4.5°2θ.

Clause 36. Crystalline thiocholesterol Form A of clause 34, having an x-ray powder diffraction pattern comprising a peak at about 8.4°2θ.

Clause 37. Crystalline thiocholesterol Form A of clause 34, having an x-ray powder diffraction pattern comprising a peak at about 10.1°2θ.

Clause 38. Crystalline thiocholesterol Form A of clause 34, having an x-ray powder diffraction pattern comprising a peak at about 12.2°2θ.

Clause 39. Crystalline thiocholesterol Form A of clause 34, having an x-ray powder diffraction pattern comprising a peak at about 15.6°2θ.

Clause 40. Crystalline thiocholesterol Form A of clause 34, having an x-ray powder diffraction pattern comprising a peak at about 16.6°2θ.

Clause 41. Crystalline thiocholesterol Form A of clause 34, having an x-ray powder diffraction pattern comprising a peak at about 17.9°2θ.

Clause 42. Crystalline thiocholesterol Form A of clause 34, having an x-ray powder diffraction pattern comprising a peak at and about 19.7°2θ.

Clause 43. Crystalline thiocholesterol Form B.

Clause 44. Crystalline thiocholesterol Form B of clauses 1 or 43, having an x-ray powder diffraction pattern comprising a peak at about 5.7°2θ.

Clause 45. Crystalline thiocholesterol Form B of clause 44, having an x-ray powder diffraction pattern further comprising a peak at about 8.2°2θ.

Clause 46. Crystalline thiocholesterol Form B of clause 44, having an x-ray powder diffraction pattern further comprising a peak at about 14.3°2θ.

Clause 47. Crystalline thiocholesterol Form B of clause 43, having an x-ray powder diffraction pattern comprising a peak at about 5.7°2θ and one or more peaks at about 8.2°2θ, at about 11.1°2θ, at about 12.3°2θ, at about 13.0°2θ, at about 14.3°2θ, at about 15.6°2θ, at about 16.2°2θ, and at about 18.8°2θ.

Clause 48. Crystalline thiocholesterol Form B of clause 47, having an x-ray powder diffraction pattern comprising a peak at about 8.2°2θ.

Clause 49. Crystalline thiocholesterol Form B of clause 47, having an x-ray powder diffraction pattern comprising a peak at about 11.1°2θ.

Clause 50. Crystalline thiocholesterol Form B of clause 47, having an x-ray powder diffraction pattern comprising a peak at about 12.3°2θ.

Clause 51. Crystalline thiocholesterol Form B of clause 47, having an x-ray powder diffraction pattern comprising a peak at about 13.0°2θ.

Clause 52. Crystalline thiocholesterol Form B of clause 47, having an x-ray powder diffraction pattern comprising a peak at about 14.3°2θ.

Clause 53. Crystalline thiocholesterol Form B of clause 47, having an x-ray powder diffraction pattern comprising a peak at about 15.6°2θ.

Clause 54. Crystalline thiocholesterol Form B of clause 47, having an x-ray powder diffraction pattern comprising a peak at about 16.2°2θ.

Clause 55. Crystalline thiocholesterol Form B of clause 47, having an x-ray powder diffraction pattern comprising a peak at about 18.8°2θ.

Clause 56. Crystalline thiocholesterol Form B of clause 47, having an x-ray powder diffraction pattern comprising a peak at about 8.2°2θ, a peak at about 14.3°2θ, and a peak at about 16.2°2θ.

Clause 57. Crystalline thiocholesterol Form B of clause 47, having an x-ray powder diffraction pattern comprising a peak at about 8.2°2θ, a peak at about 11.1°2θ, a peak at about 13.0°2θ, and a peak at about 14.3°2θ.

Clause 58. Crystalline thiocholesterol Form C.

Clause 59. Crystalline thiocholesterol Form C of clause 1 or 58, having an x-ray powder diffraction pattern comprising a peak at about 2.6°2θ.

Clause 60. Crystalline thiocholesterol Form C of clause 58 having an x-ray powder diffraction pattern comprising a peak at about 2.6°2θ and one or more peaks at about 5.3°2θ, at about 7.2°2θ, at about 14.6°2θ, and at about 14.9°2θ.

Clause 61. Crystalline thiocholesterol Form C of clause 58 having an x-ray powder diffraction pattern comprising a peak at about 7.2°2θ.

Clause 62. Crystalline thiocholesterol Form C of clause 60 having an x-ray powder diffraction pattern comprising a peak at about 5.3°2θ.

Clause 63. Crystalline thiocholesterol Form C of clause 60 having an x-ray powder diffraction pattern comprising a peak at about 7.2°2θ.

Clause 64. Crystalline thiocholesterol Form C of clause 60 having an x-ray powder diffraction pattern comprising a peak at about 14.6°2θ.

Clause 65. Crystalline thiocholesterol Form C of clause 60 having an x-ray powder diffraction pattern comprising a peak at about 14.9°2θ.

Clause 66. Crystalline thiocholesterol Form C of clause 58 having an x-ray powder diffraction pattern comprising a peak at about 7.2°2θ and one or more peaks at about 5.3°2θ, at about 14.6°2θ, and at about 14.9°2θ.

Clause 67. Crystalline thiocholesterol Form C of clause 58 having an x-ray powder diffraction pattern comprising a peak at about 7.2°2θ and a peak at about 5.3°2θ.

Clause 68. Crystalline thiocholesterol Form C of clause 58 having an x-ray powder diffraction pattern comprising a peak at about 7.2°2θ and a peak at about 14.6°2θ.

Clause 69. Crystalline thiocholesterol Form C of clause 58 having an x-ray powder diffraction pattern comprising a peak at about 7.2°2θ and a peak at about 14.9°2θ.

Clause 70. Crystalline thiocholesterol of any one of clauses 1-42, having a melting point temperature onset of about 90° C.

Clause 71. The crystalline thiocholesterol of clause 70, wherein the melting point temperature onset is measured by differential scanning calorimetry.

Clause 72. Crystalline thiocholesterol of any one of clauses 1-42 and 70-71 having an orthorhombic crystal system.

Clause 73. The crystalline thiocholesterol of clause 72, having space group P2₁2₁.

Clause 74. The crystalline thiocholesterol of any one of clauses 1-42 and 70-73 having a density of about 1.048 g/cc.

Clause 75. The crystalline thiocholesterol of any one of clauses 1-42 and 70-74 having the following approximate unit cell parameters: a=about 9.13 Å, b=about 23.81 Å, c=about 35.21 Å, α=90°, β=90°, γ=90°.

Clause 76. The crystalline thiocholesterol of any one of clauses 1-42 and 70-75 having a unit cell volume of about 7654 Å³.

Clause 77. Crystalline thiocholesterol of any one of clauses 1 and 43-57, having a melting point temperature onset of about 80° C.

Clause 78. The crystalline thiocholesterol of clause 77, wherein the melting point temperature onset is measured by differential scanning calorimetry.

Clause 79. Crystalline thiocholesterol of any one of clauses 1, 43-57 and 77-78 having a monoclinic crystal system.

Clause 80. The crystalline thiocholesterol of clause 79, having space group P2₁.

Clause 81. The crystalline thiocholesterol of any one of clauses 1, 43-57 and 77-80 having a density of about 1.032 g/cc.

Clause 82. The crystalline thiocholesterol of any one of clauses 1, 43-57, and 77-81 having the following approximate unit cell parameters: a =about 11.04 Å, b=about 7.56 Å, c=about 16.03 Å, α=90°, β=104.24°, γ=90°.

Clause 83. The crystalline thiocholesterol of any one of clauses 1, 43-57, and 77-82 having a unit cell volume of about 1296 Å³.

Clause 84. Substantially pure crystalline thiocholesterol Form A.

Clause 85. Substantially pure crystalline thiocholesterol Form A of any one of clauses 1-42 and 70-76.

Clause 86. Substantially pure crystalline thiocholesterol Form B.

Clause 87. Substantially pure crystalline thiocholesterol Form B of any one of clauses 1, 43-57 and 77-83.

Clause 88. Substantially pure crystalline thiocholesterol Form C.

Clause 89. Substantially pure crystalline thiocholesterol Form C of any one of clauses 1 and 58-69.

Clause 90. Stable crystalline thiocholesterol Form A.

Clause 91. The stable crystalline thiocholesterol Form A of clauses 1-42, 70-76, and 84-85.

Clause 92. Stable crystalline thiocholesterol Form B.

Clause 93. The stable crystalline thiocholesterol Form B of clauses 1, 43-57, 77-83, and 86-87.

Clause 94. Stable crystalline thiocholesterol Form C.

Clause 95. The stable crystalline thiocholesterol Form C of clauses 1, 58-69, and 88-89.

Clause 96. A method for preparing thiocholesterol, the method comprising: halogenating cholesterol to produce 3β-halo-5-cholestene; contacting the 3β-halo-5-cholestene with a thiourea to produce a cholestene isothiouronium salt; and
hydrolyzing the isothiouronium salt to generate thiocholesterol.

Clause 97. The method of clause 96, wherein halogenating cholesterol comprises contacting cholesterol with an organophosphorus compound and halogen source.

Clause 98. The method of clause 97, wherein the organophosphorus compound is triphenylphosphine.

Clause 99. The method of clause 97, wherein the organophosphorus compound is 1,2-bis(diphenylphosphino)ethane (DPPE).

Clause 100. The method of any one of clauses 97-99, wherein cholesterol is contacted with a catalytic amount of the organophosphorus compound.

Clause 101. The method of any one of clauses 97-99, wherein cholesterol is contacted with a stoichiometric amount of the organophosphorus compound.

Clause 102. The method of any one of clauses 97-101, wherein the halogen source comprises an alkyl halide.

Clause 103. The method of clause 102, wherein the halogen source comprises an alkyl halide selected from the group consisting of carbon tetrachloride, carbon tetrabromide and carbon tetraiodide.

Clause 104. The method of clause 103, wherein the halogen source comprises carbon tetrabromide.

Clause 105. The method of any one of clauses 97-101, wherein the halogen source comprises a metal halide.

Clause 106. The method of claim 105, wherein the halogen source comprises lithium bromide.

Clause 107. The method of any one of clauses 96-106, wherein the thiourea comprises substituted thiourea.

Clause 108. The method of any one of clauses 96-106, wherein the thiourea comprises an unsubstituted thiourea.

Clause 109. The method of any one of clauses 96-108, wherein hydrolyzing the isothiouronium salt comprises:
contacting the cholestene isothiouronium salt with a base to produce a 5-cholestene-3β-thiol salt; and
contacting 5-cholestene-3β-thiol salt with an acid to generate thiocholesterol.

Clause 110. The method of clause 109, wherein the base comprises a metal hydroxide.

Clause 111. The method of clause 110, wherein the base comprises sodium hydroxide.

Clause 112. The method of any one of clauses 109-111, wherein the acid comprises hydrochloric acid.

Clause 113. A process for preparing crystalline thiocholesterol Form A comprising: (a) treating cholesterol with carbon tetrabromide and triphenylphosphine to provide (3S,8S,9S,10R,13R,14S,17R)-3-Bromo-17-R1R)-1,5-dimethylhexyl1-10,13-dimethyl-2,3,4,7,8,9,11,12,14,15,16,17-dodecahydro-1H-cyclopenta[a]phenanthrene; (b) treating the (3S,8S,9S,10R,13R,14S,17R)-3-Bromo-17-R1R)-1,5-dimethylhexyl1-10,13-dimethyl-2,3,4,7,8,9,11,12,14,15,16,17-dodecahydro-1H-cyclopenta[a]phenanthrene with thiourea to provide [amino-[ [(3S,8S,9S ,10R,13R,14S,17R)-17-[(1R)-1,5-dimethylhexyl]-10,13-dimethyl-2,3,4,7,8,9,11,12,14,15,16,17-dodecahy dro-1H-cyclopenta[a] phenanthren-3-yl]sulfanyl]methylene]ammonium;bromide; and (c) hydrolyzing, then concentrating, the [amino-[[(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfanyl]methylene]ammonium;bromide to provide the crystalline thiocholesterol Form A.

Clause 114. The process of clause 113 wherein step (a) is performed in a suitable solvent, wherein the suitable solvent is optionally dichloromethane.

Clause 115. The process of any one of clauses 113-114 further comprising, between steps (a) and (b), the steps of diluting the mixture obtained from step (a) with hexanes and filtering to provide a filtrate comprising the (3S,8S,9S,10R,13R,14S,17R)-3-bromo-17-[(1R)-1,5-dimethylhexyl]-10,13-dimethyl-2,3,4,7,8,9,11,12,14,15,16,17-dodecahydro-1H-cyclopenta[a] phenanthrene.

Clause 116. The process of clause 115, wherein the filtering is conducted with a pad of silica gel.

Clause 117. The process of any one of clauses 115-116, wherein the filtrate is concentrated.

Clause 118. The process of any one of clauses 113-117, wherein in step (b) the (3 S,8S,9S,10R,13R,14S,17R)-3-Bromo-17-R1R)-1,5-dimethylhexyl1-10,13-dimethyl-2,3,4,7,8,9,11,12,14,15,16,17-dodecahydro-1H-cyclopenta[a]phenanthrene is treated with a second suitable solvent.

Clause 119. The process of clause 118, wherein the second suitable solvent is isopropanol.

Clause 120. The process of any one of clauses 113-119, wherein the reaction mixture in step (b) is refluxed.

Clause 121. The process of clause 120, wherein, after refluxing, the resulting mixture is cooled and filtered, and optionally then washed and dried, to provide the [amino-[[(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfanyl]methylene]ammonium; bromide.

Clause 122. The process of clause 121, wherein the mixture is cooled to about 0° C.

Clause 123. The process of any one of clauses 113-122, wherein the hydrolyzing in step (c) comprises treating the [amino-[[(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfanyl]methylene]ammonium;bromide with a base and then neutralizing with an acid.

Clause 124. The process of clause 123, wherein the base is NaOH and/or the acid is HCl.

Clause 125. The process of any one of clauses 113-124, wherein the concentrating in step (c) comprises one or more of: (i) removing any aqueous phase by extraction from an organic phase containing the thiocholesterol; (ii) washing, drying, filtering and/or concentrating an organic phase containing the thiocholesterol; (iii) filtering and eluting the residue obtained from (ii) to obtain a filtrate containing the thiocholesterol; and (iv) concentrating an organic phase containing the thiocholesterol, which optionally is the filtrate obtained in (iii), to provide the crystalline thiocholesterol Form A.

Clause 126. Crystalline thiocholesterol Form A made by the process of any one of clauses 113-124.

Clause 127. A process for preparing crystalline thiocholesterol Form B comprising the steps of treating solid thiocholesterol with a suitable solvent to form a solution and evaporating the solution.

Clause 128. The process of clause 127, wherein the solid thiocholesterol is crystalline thiocholesterol Form A.

Clause 129. The process of clause 127 or 128, wherein the suitable solvent is dichloromethane.

Clause 130. Crystalline thiocholesterol Form B made by the process of any one of clauses 127-129.

Clause 131. A process for preparing crystalline thiocholesterol Form C comprising the steps of treating solid thiocholesterol with a suitable solvent and evaporating the solvent.

Clause 132. The process of clause 131, wherein the solid cholesterol is crystalline thiocholesterol Form A.

Clause 133. The process of clause 131 or 132, wherein the suitable solvent is diethyl ether.

Clause 134. Crystalline thiocholesterol Form C made by the process of any one of clauses 114-116.

Clause 135. A mixture of one or more of crystalline thiocholesterol Form A, crystalline thiocholesterol Form B, and crystalline thiocholesterol Form C.

Clause 136. A mixture of one or more of crystalline thiocholesterol Form A of clauses 1-42, 70-76, 84-85, 89-91, and 0126; crystalline thiocholesterol Form B of clauses 1, 43-57, 77-83, 86-87, 92-93, and 113; and crystalline thiocholesterol Form C of clauses 1, 58-69, 88-89, 94-95, and 134.

Clause 137. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis, comprising administering to a patient in need thereof an effective amount of crystalline thiocholesterol.

Clause 138. The method of clause 137 wherein the crystalline thiocholesterol is one or more of crystalline thiocholesterol Form A, crystalline thiocholesterol Form B, and/or crystalline thiocholesterol Form C.

Clause 139. The method of clause 137-138 wherein the crystalline thiocholesterol is one or more of crystalline thiocholesterol Form A of clauses 1-42, 70-76, 84-85, 89-91, and 109; crystalline thiocholesterol Form B of clauses 1, 43-57, 77-83, 86-87, 92-93, and 113; and crystalline thiocholesterol Form C of clauses 1, 58-69, 88-89, 94-95, and 134.

Clause 140. A pharmaceutical composition comprising crystalline thiocholesterol and at least one pharmaceutically acceptable excipient.

Clause 141. A pharmaceutical composition comprising one or more of crystalline thiocholesterol Form A of clauses 1-42, 70-76, 84-85, 89-91, and 126; crystalline thiocholesterol Form B of clauses 1, 43-57, 77-83, 86-87, 92-93, and 130; and crystalline thiocholesterol Form C of clauses 1, 58-69, 88-89, 94-95, and 134, and at least one pharmaceutically acceptable excipient.

Clause 142. The method of any one of clauses 137-139 wherein the crystalline thiocholesterol is in the form of a pharmaceutical composition further comprising at least one pharmaceutically acceptable excipient.

Clause 143. Use of crystalline thiocholesterol as defined in any one of clauses 1-134, a mixture as defined in clauses 135 or 136 or a pharmaceutical composition as defined in clauses 140 or 141 in the manufacture of a medicament for use in the treatment or prevention of one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

Clause 144. Crystalline thiocholesterol as defined in any one of clauses 1-134, or a mixture as defined in clauses 135 or 136 or a pharmaceutical composition as defined in clauses 140 or 141 for use as a medicament.

Clause 145. Crystalline thiocholesterol as defined in any one of clauses 1-134, a mixture as defined in clauses 135 or 136 or a pharmaceutical composition as defined in clauses 140 or 141 for use in a method for the treatment or prevention of one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

Clause 146. Crystalline thiocholesterol of clause 1 having an x-ray powder diffraction pattern comprising a peak between about 5.3°2θ and about 5.7°2θ.

Clause 147. Crystalline thiocholesterol of clause 1 having an x-ray powder diffraction pattern comprising a peak between about 14.9°2θ and about 15.0°2θ.

Clause 132. Crystalline thiocholesterol of clause 1 having an x-ray powder diffraction pattern comprising a peak between about 15.5°2θ and about 15.6°2θ.

### EXAMPLES

### Example 1—Use of Crystalline Thiocholesterol Form A

Thiocholesterol was used screening activities and was prepared in accordance with Example 2. The lot was identified as crystalline thiocholesterol Form A by x-ray powder diffraction (XRPD). An XRPD diffractogram of crystalline thiocholesterol Form A is found in FIG. 1.

### Example 2—Preparation of Crystalline Thiocholesterol Form A

Carbon tetrabromide (25.7 g, 77.6 mmol) was added to a mixture of cholesterol (20.0 g, 51.7 mmol) and triphenylphosphine (20.4 g, 77.6 mmol) in dichloromethane (200 mL) at 0° C. The mixture was stirred at room temperature for 10 h. The mixture was diluted with hexanes (100 mL) and filtered through a pad of silica gel. The silica pad was further washed with hexanes. The filtrate was concentrated to provide (3S,8S,9S,10R,13R,14S,17R)-3-bromo-17-[(1R)-1,5-dimethylhexyl]-10,13-dimethyl-2,3,4,7,8,9,11,12,14,15,16,17-dodecahydro-1H-cyclopenta[a]phenanthrene (33.0 g, 70.3% pure, 100%). (3S,8S,9S, 10R,13R,14S,17R)-3-Bromo-17-[(1R)-1,5-dimethylhexyl]-10,13-dimethyl-2,3,4,7,8,9,11,12,14,15,16,17-dodecahydro-1H-cyclopenta[a]phenanthrene was diluted with iPrOH (170 mL). Thiourea (19.7 g, 258 mmol) was added to the mixture, and the solution was refluxed for 48 h. Ice (20.0 g) was added to the mixture at 0° C., and the mixture was filtered. The solid was washed with a 2:1 mixture of acetone/water (400 mL), acetone (10.0 mL), and hexanes (200 mL) and dried under reduced pressure to form [amino-[[(3S,8S,9S, 10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfanyl]methylene]ammonium;bromide as solid (25.7 g, 95%). 1H NMR (500 MHz, DMSO) δ 9.01 (d, J=79.8 Hz, 4H), 5.40 (s, 1H), 3.63 (t, J=13.0 Hz, 1H), 2.34 (td, J=12.6, 7.3 Hz, 2H), 2.03-1.71 (m, 5H), 1.71-0.75 (m, 33H), 0.66 (s, 3H).

NaOH (5.86 g, 147 mmol) was added to a solution of [amino-[[(3 S,8 S,9S,10R,13R,14S,17R)-17-[(1R)-1,5-dimethylhexyl]-10,13-dimethyl-2,3,4,7,8,9,11,12,14,15,16,17-dodecahy dro-1H-cyclopenta[a] phenanthren-3-yl]sulfanyl]methylene]ammonium;bromide (25.7 g, 48.9 mmol) in EtOH (400 mL) at 0° C. The mixture was stirred at 22° C. for 4 h and was neutralized (pH 7) with HCl (1 M in water). The aqueous phase was extracted with hexanes (3×300 mL), and the combined organic phases were washed with water (300 mL), dried (Na2SO4), filtered, and concentrated. The residue was filtered through silica gel, eluting with hexanes and EtOAc (9:1), and the filtrate was concentrated to provide the title compound as a solid (14.1 g, 71%). 1H NMR (500 MHz, CDCl3) δ 5.25 (d, J=4.6 Hz, 1H), 2.69-2.54 (m, 1H), 2.24 (d, J=7.6 Hz, 2H), 2.01-1.70 (m, 5H), 1.60-0.74 (m, 34H), 0.60 (s, 3H).

### Example 3—Preparation of Crystalline Thiocholesterol Form B

A clear solution containing 38.3 mg thiocholesterol from Example 2 in 1 mL of dichloromethane (DCM) was filtered through a 0.2-μm nylon filter into a 1-dram scintillation vial. The vial opening was sealed with aluminum foil, pierced once with a pin, and the solution allowed to evaporate to dryness at ambient conditions.

### Example 4—Preparation of Crystalline Thiocholesterol Form C

A clear solution containing 46.1 mg of thiocholesterol lot from Example 2 in 1 mL of diethyl ether was filtered through a 0.2-μm nylon filter into a 1-dram scintillation vial. The vial opening was sealed with aluminum foil, pierced once with a pin, and the solution allowed to evaporate to dryness at ambient conditions.

### Example 5—Screening Methods

### Cooling and Slow Cools

Solutions were prepared in the selected solvent or solvent/anti-solvent system. These solutions were chilled below room temperature within a refrigerator for varying lengths of time in an attempt to induce nucleation. The presence or absence of solids was noted. Upon observation of solids, in quantities sufficient for analysis, isolation of material was conducted. If insufficient quantities were present, further cooling was performed in a freezer. Samples were either isolated for analysis wet or as dry powders.

### Fast Evaporation

Solutions were prepared in selected solvents and agitated between aliquot additions to assist in dissolution. Once a mixture reached complete dissolution, as judged by visual observation, the solution was filtered through a 0.2-μm nylon filter and allowed to evaporate at ambient temperature in an uncapped vial or at ambient under nitrogen. The solids that formed were isolated for evaluation.

### Heating

Samples were heated on hot plate to evaluate potential heat-induced transformations and to recrystallize material from the melt.

### Slurry

Solutions were prepared by adding enough solids to a given solvent so that excess solids were present. The mixture was then agitated in a sealed vial at either ambient or an elevated temperature. After a given amount of time, the solids were isolated for analysis.

### Example 6—Instrumental Techniques

### Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin, and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to analysis. Samples were analyzed from −30° C. to 250° C. @ 10°/min.

### Dynamic Vapor Sorption/Desorption (DVS)

Automated vapor sorption (VS) data were collected on a Surface Measurement System DVS Intrinsic instrument. Samples were not dried prior to analysis. Sorption and desorption data were collected over a range from 5% to 95% RH at 10% RH increments under a nitrogen purge. The equilibrium criterion used for analysis was less than 0.0100% weight change in 5 minutes with a maximum equilibration time of 3 hours. Data were not corrected for the initial moisture content of the samples.

### Polarized Light Microscopy

Polarized light microscopy was performed using a Motic SMZ-168. Each sample was observed using a 10× objective at 0.75 up to 5.0× magnification with crossed polarizers.

### Proton NMR Spectroscopy

The solution NMR spectrum acquired with an Avance 600 MHz NMR Spectrometer. The sample was prepared by dissolving approximately a weighed amount of sample in CDCl3 containing TMS. The data acquisition parameters are displayed in the first plot of the spectrum in the Data section of this report.

### Thermogravimetric Analysis (TGA)

TG analysis was performed using a Mettler-Toledo TGA/DSC3 analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Each sample was heated from ambient temperature to 350° C. at 10° C./min. Although thermograms are plotted by reference temperature (x-axis), results are reported according to sample temperatures.

### Example 7—Single Crystal Data Collection on Crystalline Thiocholesterol Form A and Crystalline Thiocholesterol Form B

Standard uncertainty in this report is written in crystallographic parenthesis notation, e.g. 0.123(4) is equivalent to 0.123±0.004. Calculated x-ray powder diffraction patterns were generated for Cu radiation using MERCURY and the atomic coordinates, space group, and unit cell parameters from the single crystal structure. The atomic displacement ellipsoid diagrams were prepared using MERCURY. Atoms are represented by 50% probability anisotropic thermal ellipsoids.

### Crystalline Thiocholesterol Form A

The quality of the structure obtained is moderate, as indicated by the fit residual, R, of 0.0746 (7.46%). R-factors in the range 2%-6% are quoted to be the most reliably determined structures. While the overall quality of the structure falls outside of this range, the data quality was sufficient to determine the molecular conformation of Thiocholesterol in this structure and the contents of the asymmetric unit.

### Data Collection

A colorless block having approximate dimensions of 0.2×0.11×0.04 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 6257 reflections in the range 3.9070°<θ<72.6080°. The space group was determined by the program CRYSALISPRO to be P2₁2₁2₁ (international tables no. 19).

The data were collected to a maximum diffraction angle (2θ) of 156.188° at room temperature.

### Data Reduction

Frames were integrated with CRYSALISPRO. A total of 26719 reflections were collected, of which 14363 were unique. Lorentz and polarization corrections were applied to the data. The linear absorption coefficient is 1.165 mm⁻¹ for Cu Kα radiation. An empirical absorption correction using CRYSALISPRO was applied. Transmission coefficients ranged from 0.892 to 1.000. Intensities of equivalent reflections were averaged. The agreement factor for the averaging was 4.76% based on intensity.

### Structure Solution and Refinement

The structure was solved by direct methods using SHELXT. The remaining atoms were located in succeeding difference Fourier syntheses. The structure was refined using SHELXL-2014. Hydrogen atoms were included in the refinement but restrained to ride on the atom to which they are bonded. The structure was refined in full-matrix least-squares by minimizing the function:

Σw(|Fₒ|²−|F_{c}|²)²

where the weight, w, is defined as 1/[σ²(Fₒ²)+(0.1298P)²], where P=(Fₒ²+2F_{c}²)/3.

Scattering factors were taken from the “International Tables for Crystallography”. Of the 14363 reflections used in the refinements, only the reflections with intensities larger than twice their uncertainty [I>2σ(I)], 7471, were used in calculating the fit residual, R. The final cycle of refinement included 775 variable parameters, 0 restraints, and converged with respective unweighted and weighted agreement factors of:

R=Σ|Fₒ−F_{c}|/ΣFₒ=0.0746

R_{w}={square root over ((Σw(Fₒ²−F_{c}²)²/Σw(Fₒ²)²))}=0.1938

The standard deviation of an observation of unit weight (goodness of fit) was 0.98. The highest peak in the final difference Fourier had an electron density of 0.250 e/Å³. The minimum negative peak had a value of −0.180 e/Å³.

### Crystalline Thiocholesterol Form B

The quality of the structure obtained is high, as indicated by the fit residual, R, of 0.0591 (5.91%). R-factors in the range 2%-6% are quoted to be the most reliably determined structures.

### Data Collection

A colorless plate having approximate dimensions of 0.55×0.43×0.06 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 2717 reflections in the range 4.4160°<θ<75.0730°. The space group was determined by the program CRYSALISPRO to be P2₁ (international tables no. 4).

The data were collected to a maximum diffraction angle (2θ) of 154.616° at room temperature.

### Data Reduction

Frames were integrated with CRYSALISPRO. A total of 5888 reflections were collected, of which 3694 were unique. Lorentz and polarization corrections were applied to the data. The linear absorption coefficient is 1.146 mm⁻¹ for Cu Kα radiation. An empirical absorption correction using CRYSALISPRO was applied. Transmission coefficients ranged from 0.678 to 1.000. A secondary extinction correction was applied. The final coefficient, refined in least-squares, was 0.0055(17) (in absolute units). Intensities of equivalent reflections were averaged. The agreement factor for the averaging was 2.4% based on intensity.

### Structure Solution and Refinement

The structure was solved by direct methods using SHELXT. The remaining atoms were located in succeeding difference Fourier syntheses. The structure was refined using SHELXL-2014. Hydrogen atoms were included in the refinement but restrained to ride on the atom to which they are bonded. The structure was refined in full-matrix least-squares by minimizing the function:

Σw(|Fₒ|²−|F_{c}|²)²

where the weight, w, is defined as 1/[σ²(Fₒ²)+(0.1285P)²+(0.0103P)], where P=(Fₒ²+2F_{c}²)/3.

Scattering factors were taken from the “International Tables for Crystallography”. Of the 3694 reflections used in the refinements, only the reflections with intensities larger than twice their uncertainty [I>2σ(I)], 2478, were used in calculating the fit residual, R. The final cycle of refinement included 260 variable parameters, 1 restraints, and converged with respective unweighted and weighted agreement factors of:

R=Σ|Fₒ−F_{c}|/ΣFₒ=0.0591

R_{w}={square root over ((Σw(Fₒ²−F_{c}²)²/Σw(Fₒ²)²))}=0.1880

The standard deviation of an observation of unit weight (goodness of fit) was 1.08. The highest peak in the final difference Fourier had an electron density of 0.172 e/Å³. The minimum negative peak had a value of −0.128 e/Å³.

### Example 7—X-Ray Powder Diffraction (XRPD)

### Transmission

X-ray powder diffraction pattern was collected with a PANalytical X'Pert PRO MPD or PANalytical Empyrean diffractometer using an incident beam of Cu radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5. The data acquisition parameters are listed in the image of each pattern displayed in the Data section of this report. All images have the instrument labeled as X'Pert PRO MPD regardless of the instrument used.

### Reflection Geometry

X-ray powder diffraction patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5. The data acquisition parameters for each pattern are displayed above the image in the Data section of this report including the divergence slit (DS) and the incident-beam SS.

1. Crystalline thiocholesterol.

2. Crystalline thiocholesterol Form A.

3. Crystalline thiocholesterol Form A of claim 2, having an x-ray powder diffraction pattern comprising a peak at about 4.5°2θ.

4. Crystalline thiocholesterol Form A of any one of claims 1-2 having an x-ray powder diffraction pattern comprising two peaks at about 11.0°2θ and at about 15.0°2θ and one or more peaks at about 4.5°2θ, at about 8.4°2θ, at about 10.1°2θ, at about 12.2°2θ, at about 15.6°2θ, at about 16.6°2θ, at about 17.9°2θ, and at about 19.7°2θ.

5. Crystalline thiocholesterol Form B.

6. Crystalline thiocholesterol Form B of claim 1 or 5, having an x-ray powder diffraction pattern comprising a peak at about 5.7°2θ.

7. Crystalline thiocholesterol Form C.

8. Crystalline thiocholesterol Form C of claim 1 or 7, having an x-ray powder diffraction pattern comprising a peak at about 2.6°2θ.

9. Crystalline thiocholesterol of any one of claims 1-4, having a melting point temperature onset of about 90° C.

10. Crystalline thiocholesterol of any one of claims 1-4 and 9 having an orthorhombic crystal system.

11. The crystalline thiocholesterol of any one of claims 1-4 and 9-10 having a density of about 1.048 g/cc.

12. The crystalline thiocholesterol of any one of claims 1-4 and 9-11 having the following approximate unit cell parameters: a=about 9.13 Å, b=about 23.81 Å, c=about 35.21 Å, α=90°, β=90°, γ=90°.

13. The crystalline thiocholesterol of any one of claims 1-4 and 9-12 having a unit cell volume of about 7654 Å³.

14. Crystalline thiocholesterol of any one of claims 1 and 5-6, having a melting point temperature onset of about 80° C.

15. Crystalline thiocholesterol of any one of claims 1, 5-6 and 14 having a monoclinic crystal system.

16. The crystalline thiocholesterol of any one of claims 1, 5-6 and 14-15 having a density of about 1.032 g/cc.

17. A pharmaceutical composition comprising crystalline thiocholesterol and at least one pharmaceutically acceptable excipient.

18. A pharmaceutical composition comprising one or more of crystalline thiocholesterol Form A of claims 1-4 and 9-13; crystalline thiocholesterol Form B of claims 1, 5-6, and 14-16; and crystalline thiocholesterol Form C of claims 1 and 7-8, and at least one pharmaceutically acceptable excipient.
